(19) United States
(12) Patent Application Publication
(10) Pub. No.: US 2021/0140977 A1
(43) Pub. Date: May 13, 2021

(54) A THREE-PROTEIN PROTEOMIC BIOMARKER FOR PROSPECTIVE DETERMINATION OF RISK FOR DEVELOPMENT OF ACTIVE TUBERCULOSIS

(71) Applicants: UNIVERSITY OF CAPE TOWN, Cape Town (ZA); SEATTLE CHILDREN'S HOSPITAL DBA SEATTLE CHILDREN'S RESEARCH INSTITUTE ("SCRI"), Seattle, WA (US)

(72) Inventors: Thomas Jens Scriba, Cape Town (ZA); Adam Garth Penn-Nicholson, Geneva (CH); Daniel Edward Zak, Seattle, WA (US); Ethan Greene Thompson, Seattle, WA (US)

(21) Appl. No.: 17/048,062

(22) PCT Filed: Apr. 12, 2019
(86) PCT No.: PCT/IB2019/053025

§ 371 Date: Oct. 15, 2020

(30) Foreign Application Priority Data
Apr. 16, 2018 (ZA) 2018/02474

(51) Int. Cl. G01N 33/68; G16H 50/20; G16H 50/30; G16H 10/40

(52) U.S. Cl. CPC G01N 33/6893; G16H 50/20; G16H 50/30; G16H 10/40; G01N 2800/60; G01N 2333/525; G01N 2333/9123; G01N 2800/52; G01N 2333/4716

(57) ABSTRACT

The invention relates to a method and kit for determining a likelihood of a human subject with asymptomatic tuberculosis (TB) infection or suspected TB infection progressing to active tuberculosis disease, the method comprising detecting a presence or level of a first and a second pair of protein biomarkers selected from Complement Component 9 (C9) and Complement C1q Tumor Necrosis Factor-Related Protein 3 (C1qTNF3); and C9 and Creatine Kinase M- and B-type (CKMB) in a sample from the subject.

### FIELD OF THE INVENTION

This invention relates to a prognostic method for determining the risk of an asymptomatic human subject with latent tuberculosis (TB) infection or apparent latent TB infection and/or after suspected exposure to TB progressing to active tuberculosis disease comprising the steps of quantifying and computationally analysing relative abundances of a collection or panel of pairs of protein products (“TB proteomic biomarkers”) derived from a sample obtained from the subject. The invention further relates to a collection or panel of TB proteomic biomarker pairs that generates a proteomic signature of risk for prediction of the likelihood of an asymptomatic human subject with latent TB infection and/or after suspected exposure to TB progressing to active tuberculosis disease. Furthermore, a kit comprising protein-specific binding and detection molecules for the detection of pairs of TB proteomic biomarkers that generates a prognostic signature of risk for use with the method of the invention is described. In addition, the invention relates to a method of preventive treatment or prophylaxis for TB infection comprising the use of the prognostic method and/or the kit of the invention to select an appropriate or experimental treatment regimen or intervention for the human subject and/or to monitor the response of the human subject to the TB prophylaxis. In various additional embodiments, the invention relates to one or more devices, reagents, and/or systems for detecting and/or characterizing the likelihood of an asymptomatic human subject with latent TB infection and/or after suspected exposure to TB progressing to active tuberculosis disease.

### BACKGROUND OF THE INVENTION

Mycobacterium tuberculosis and other mycobacteria cause tuberculosis (TB). If not treated properly, TB disease can be fatal. However, not everyone infected with TB bacteria becomes sick. As a result, two TB-related conditions exist: latent TB infection and active TB disease.

Both latent TB infection and active TB disease can be treated, although the treatment regimens are different. One-quarter of the global population is latently infected with Mycobacterium tuberculosis, but only 5-10% will progress to active tuberculosis disease during their life-time, while the majority will remain healthy with latent Mycobacterium tuberculosis infection. Risk of progression from latent to active tuberculosis is associated with young or old age, immunocompromise such as HIV infection and co-morbidities such as diabetes mellitus, socio-economic and nutritional compromise, and therapy with immune modulatory agents such as tumour necrosis factor inhibitors, among others. The current vaccine to prevent TB disease is not sufficiently efficacious, while current strategies to diagnose and treat patients with active tuberculosis disease are not having an acceptable impact on the TB epidemic.

A biomarker capable of predicting progression of healthy individuals to active TB disease before the emergence of clinical symptoms may allow targeted and rapid treatment before active disease manifests, with applications to curb transmission and halt the global epidemic. While transcriptomic biomarkers have shown potential, a protein-based biomarker with comparable performance could offer a cheaper alternative suitable to point-of-care diagnostic devices.

### SUMMARY OF THE INVENTION

According to a first aspect of the invention there is provided a method of determining the likelihood of a human subject with asymptomatic tuberculosis (TB) infection or suspected TB infection progressing to active tuberculosis disease comprising detecting the presence or level of a first and a second pair of protein biomarkers selected from Complement Component 9 (C9) and Complement C1q Tumor Necrosis Factor-Related Protein 3 (C1qTNF3); and C9 and Creatine Kinase M- and B-type (CKMB) in a sample from the subject.

In one embodiment, the method comprises the steps of:
(a) providing the sample from a human subject with asymptomatic TB infection or suspected TB infection;
(b) quantifying and computationally analysing relative abundances of a 3 protein pair-ratio (3PR) signature consisting of a first and a second pair of proteins selected from Complement Component 9 (C9) and Complement C1q Tumor Necrosis Factor-Related Protein 3 (C1qTNF3); and C9 and Creatine Kinase M- and B-type (CKMB); and
(c) computing a prognostic score of the risk of the subject developing active TB disease, thus classifying the subject as “progressor” or “non-progressor”, wherein a prognostic score of “progressor” indicates that the subject with asymptomatic TB infection or suspected TB infection is likely to progress to active tuberculosis disease.

The asymptomatic tuberculosis infection or suspected TB infection may be latent TB infection in the subject, apparent latent TB infection in the subject, suspected active TB disease in the subject, or after exposure of the subject to an infectious person with TB. For example, the TB infection may be Mycobacterium tuberculosis (Mtb), Mycobacterium bovis and/or Mycobacterium africanum infection.

The computational analysis may comprise the computation of a log ratio:

r=log_2(concentration protein 1/concentration protein 2)

for the first and second pair of proteins from a sample and the use of a score table that has been calculated by analysis of a prospective TB risk cohort to convert the computed log ratio for each pair of proteins into a score, followed by calculation of the mean final score from both pairs of proteins from the sample wherein the mean final score is predictive of the likelihood of the subject with asymptomatic TB infection or suspected TB infection progressing to active tuberculosis disease.

In particular, the computational analysis may comprise the steps of:
(i) quantifying the protein concentration of the three proteins C9, C1qTNF3 and CK-MB;
(ii) computing the difference in concentration between the protein pairs C9 and C1qTNF3 (pair 1) and C9 and CK-MB (pair 2) to generate a log-transformed ratio of expression for each pair;
(iii) comparing the log-transformed ratio of expression for pair 1 and pair 2 to the closest minimal ratios listed in Table 1 and Table 2 respectively by finding the minimal ratio in the first column of the respective table that is greater than or equal to the computed log-transformed ratio;
(iv) assigning a corresponding numerical score in the second column of the respective table to the computed log-transformed ratio for pair 1 and pair 2, wherein if the computed log-transformed ratio is greater than all of the ratios in column 1 of the respective table, assigning a numerical score of 1 to the computed log-transformed ratio;
(v) determining the final score for pair 1 and pair 2 by computing the average value of the numerical scores generated from both pair 1 and pair 2, wherein the final score is predictive of the likelihood of the subject with asymptomatic TB infection or suspected TB infection progressing to active tuberculosis disease.

For example, the default threshold for progressor vs non-progressors may be 0.5 (50%). However, it is to be appreciated that this threshold may be optimized to greater than 0.5 or less than 0.5 depending on the population to be tested and the epidemiologic setting in which the subject resides.

The analysis of the prospective TB risk cohort may take into account the time prior to tuberculosis diagnosis at which each sample of biological materials was obtained from the subjects in the prospective TB risk cohort.

The method may be indicative of and/or diagnostic for an asymptomatic TB infection or suspected TB infection that is likely to develop into active TB disease within 30 days, 45 days, 60 days, 90 days, 120 days, 180 days, 270 days, 360 days, 450 days, or 540 days. The subject may be identified as being likely to progress to active TB disease greater than 2 years from diagnosis if a prognostic score of “progressor” is computed.

The method may further comprise detecting the level of one or more biomarkers that are indicative of one or more of: the presence of latent TB infection, the presence of active TB disease, the strain of TB, the antibiotic resistance or sensitivity of TB, and the presence of other diseases.

The method may comprise contacting the protein biomarkers of the sample from the subject with a set of biomarker capture reagents, wherein each biomarker capture reagent of the set of biomarker capture reagents specifically binds to a different biomarker being detected.

For example, the biomarker capture reagent may be an antibody or an aptamer. The capture reagent may be labeled with an indicator molecule such as a fluorescent, chemiluminescent, radioactive, or chromogenic molecule.

The protein biomarker concentrations may be quantified by techniques such as by lateral flow technology, enzyme-linked immunosorbent assay (ELISA), surface plasmon resonance, surface acoustic waves, mass spectrometry, infrared spectroscopy, Raman spectroscopy, atomic force microscopy, scanning tunneling microscopy, electrochemical detection methods, nuclear magnetic resonance, quantum dots, or by any equivalent method for protein quantification known to those skilled in the art. In particular the technique may be a point-of-care technique.

The sample may be a biological material. The biological material may be selected from any one or more of a blood sample, a blood plasma sample, a blood serum sample derived from clotted whole blood, a blood protein sample, a sputum sample, a sputum protein sample, a urine sample, a saliva sample, a cerebrospinal fluid sample, a pleural effusion sample, a pericardial effusion sample, a tissue aspirate or biopsy sample, or any other fluid sample derived from a human.

The subject may have been treated for TB disease.

According to a further embodiment of the invention there is provided a kit comprising at least three protein biomarker capture reagents, wherein each protein biomarker capture reagent specifically binds to a protein target selected from C9, C1qTNF3 and CK-MB, and wherein each protein biomarker capture reagent specifically binds to a different target protein.

The capture reagents preferably comprise three aptamers or antibodies, wherein each aptamer or antibody specifically binds to a different target protein.

The capture reagents may be labeled with an indicator molecule including a fluorescent, chemiluminescent, radioactive, or chromogenic molecule.

The kit may further comprise one or more of: a solid support, instructions for use of the kit, a computer system or software to analyze data, and additional reagents for quantifying the levels of the protein biomarkers including reagents for processing a biological sample including solubilization buffers, detergents, washes, or buffers, and buffers, blocking agents, mass spectrometry matrix materials, antibody capture agents, detectable labels, signal generating material, positive control samples and negative control samples.

In particular, the instructions for use of the kit may include instructions for monitoring a latent TB infection in a subject for the likelihood of the latent TB infection transitioning to active TB disease comprising:
a) quantifying and computationally analysing relative abundances of a 3 protein pair-ratio (3PR) signature consisting of a first and a second pair of proteins selected from Complement Component 9 (C9) and Complement C1q Tumor Necrosis Factor-Related Protein 3 (C1qTNF3), and C9 and Creatine Kinase M- and B-type (CKMB); and
b) computing a prognostic score of the risk of the subject developing active TB disease, thus classifying the subject as “progressor” or “non-progressor”, wherein a prognostic score of “progressor” indicates that the subject with asymptomatic TB infection or suspected TB infection is likely to progress to active tuberculosis disease.

In particular, the computationally analysis may comprise:
(i) the computation of a log ratio:

r=log_2(concentration protein 1/concentration protein 2)
for the first and second pair of proteins from the sample; and
(ii) use of a score table that has been calculated by analysis of a prospective TB risk cohort to convert the computed log ratio for each pair of proteins into a score;
(iii) followed by calculation of the mean final score from both pairs of proteins from the sample,
wherein the mean final score is predictive of the likelihood of the subject with asymptomatic TB infection or suspected TB infection progressing to active tuberculosis disease.

For example, the computationally analysis may comprise:
A. quantifying the protein concentration of the three proteins C9, C1qTNF3 and CK-MB;
B. computing the difference in concentration between the protein pairs C9 and C1qTNF3 (pair 1) and C9 and CK-MB (pair 2) to generate a log-transformed ratio of expression for each pair;
C. comparing the log-transformed ratio of expression for pair 1 and pair 2 to the closest minimal ratios listed in Table 1 and Table 2 respectively by finding the minimal ratio in the first column of the respective table that is greater than or equal to the computed log-transformed ratio;
D. assigning a corresponding numerical score in the second column of the respective table to the computed log-transformed ratio for pair 1 and pair 2, wherein if the computed log-transformed ratio is greater than all of the ratios in column 1 of the respective table, assigning a numerical score of 1 to the computed log-transformed ratio; and
E. determining the final score for pair 1 and pair 2 by computing the average value of the numerical scores generated from both pair 1 and pair 2,
wherein the final score is predictive of the likelihood of the subject with asymptomatic TB infection or suspected TB infection progressing to active tuberculosis disease.

The default threshold for progressor versus non-progressors may be 0.5 (i.e. 50%), greater than 0.5 (i.e. 50%), or less than 0.5 (i.e. 50%), depending on the population to be tested and the epidemiologic setting in which the subject resides.

The computer system or software to analyze data may comprise computer readable instructions for performing each of the steps of the computational analysis.

The prognostic score of “progressor” may be indicative of one or more of: the presence of latent TB infection, the presence of active TB disease, the strain of TB, the antibiotic resistance or sensitivity of TB, and the presence of non-TB diseases.

The subject may be identified as being likely to transition to active TB disease within 30 days, 45 days, 60 days, 90 days, 120 days, 180 days, 270 days, 360 days, 450 days, or 540 days if a prognostic score of “progressor” is computed.

The instructions for use may further direct the use of the kit with any one or more of techniques including lateral flow technology, enzyme-linked immunosorbent assay (ELISA), surface plasmon resonance, surface acoustic waves, mass spectrometry, infrared spectroscopy, Raman spectroscopy, atomic force microscopy, scanning tunneling microscopy, electrochemical detection methods, nuclear magnetic resonance or quantum dots.

According to a further embodiment of the invention there is provided a panel of three aptamers or antibodies for use with the method or kit of the invention, wherein each aptamer or antibody specifically binds to the protein biomarkers C9, C1qTNF3 or CK-MB respectively. Each aptamer or antibody may be labeled with an indicator molecule such as a fluorescent, chemiluminescent, radioactive, or chromogenic molecule. The panel may be for use in a method for determining the risk of a human subject with asymptomatic TB infection or suspected TB infection progressing to active tuberculosis disease.

According to a further embodiment of the invention there is provided a composition comprising target proteins in a sample from a subject and three protein biomarker capture reagents, wherein each protein biomarker capture reagent specifically binds to a target protein selected from a first and a second pair of protein biomarkers selected from Complement Component 9 (C9) and Complement C1q Tumor Necrosis Factor-Related Protein 3 (C1qTNF3); and C9 and Creatine Kinase M- and B-type (CKMB), and wherein each protein biomarker capture reagent specifically binds a different target protein.

The at least one biomarker capture reagent may include an antibody or aptamer.

The sample may be one or more biological material sample(s) derived from a human, including a blood sample, a blood plasma sample, a blood serum sample derived from clotted whole blood, a blood protein sample, a sputum sample, a sputum protein sample, a urine sample, a saliva sample, a cerebrospinal fluid sample, a pleural effusion sample, a pericardial effusion sample, a tissue aspirate, or a biopsy sample.

The composition may be for use in a method for determining the risk of a human subject with asymptomatic TB infection or suspected TB infection progressing to active tuberculosis disease, in particular with the method of the invention, or the kit of the invention.

According to a further aspect of the invention, there is provided a method of treatment of a subject comprising the steps of (i) determining the risk of a human subject with asymptomatic TB infection or suspected TB infection progressing to active tuberculosis disease with the use of the method or the use of the kit or with the use of the panel of aptamers or antibodies of the invention, followed by (ii) prophylactic TB treatment of the subject when the subject is identified as having a risk of progression to active tuberculosis disease. The method may comprise a further step of determining the risk of the human subject to progress to active tuberculosis after the prophylactic treatment. The method may further comprise a step of on-going monitoring of human subjects identified as not having a risk of progression to active tuberculosis disease with the prognostic method or the use of the kit or with the use of the panel of aptamers or antibodies of the invention.

According to a further aspect of the invention, there is provided a method of monitoring a subject for successful prophylactic or therapeutic treatment against TB infection, or risk of recurrence of TB disease after treatment, comprising the steps of (i) determining the risk of progression to active tuberculosis disease in the subject with the method or the use of the kit or with the use of the panel of aptamers or antibodies of the invention prior to the subject undergoing prophylactic or therapeutic treatment for TB; (ii) prophylactic or therapeutic treatment of the subject for TB; and (iii) repeating the step of determining the risk of progression to active tuberculosis disease in the subject with the method or the use of the kit or with the use of the panel of aptamers or antibodies of the invention, wherein a decrease in the risk of progression after treatment compared to prior to treatment is indicative of the efficacy of the prophylactic or therapeutic treatment.

According to a further aspect of the invention, there is provided a method of reducing the incidence of active TB or preventing active TB in a subject comprising the steps of (i) determining the risk of a human subject with asymptomatic TB infection or suspected TB infection progressing to active tuberculosis disease with the method or the use of the kit or with the use of the panel of aptamers or antibodies of the invention, followed by (ii) prophylactic TB treatment of the subject when the subject is identified as having a risk of progression to active tuberculosis disease. The method may further comprise a step of on-going monitoring of human subjects identified as not having a risk of progression to active tuberculosis disease with the method or the use of the kit or with the use of the panel of aptamers or antibodies of the invention.

According to a further aspect of the invention, there is provided a method of reducing the mortality rate due to active TB comprising the steps of (i) determining the risk of a human subject with asymptomatic TB infection or suspected TB infection progressing to active tuberculosis disease with the method or the use of the kit or with the use of the panel of aptamers or antibodies of the invention, followed by (ii) prophylactic TB treatment of the subject when the subject is identified as having a risk of progression to active tuberculosis disease. The method may further comprise a step of on-going monitoring of human subjects identified as not having a risk of progression to active tuberculosis disease with the method or the use of the kit or with the use of the panel of aptamers or antibodies of the invention.

The TB treatment may include any one or more of: isoniazid, rifampicin, rifapentine, ethambutol, pyrazinamide, or any other approved or novel prophylactic or therapeutic TB treatment, vaccine or intervention regimen for a subject.

The method may further comprise performing one or more additional tests for progression of TB infection known to those skilled in the art including QuantiFERON® TB Gold In-Tube test, QuantiFERON® TB Gold Plus test, tuberculin skin test, TB GeneXpert, Xpert MTB/RIF® or other PCR tests, sputum liquid or solid medium culture, sputum smear microscopy, urine metabolite test, chest x-ray and the like on the subject.

### BRIEF DESCRIPTION OF THE DRAWINGS

FIG. 1 shows a graphical representation of pair-wise structure of the 3 protein pair-ratio (3PR) signature. Proteins that are expressed at higher levels in TB progressors, compared to non-progressors, is a filled box. Proteins expressed at levels lower in progressors than non-progressors are open boxes.

FIG. 2 shows Receiver Operator Characteristic area under the curve (ROC-AUC) analysis of the 3PR signature for all Adolescent Cohort Study (ACS) progressor and nonprogressor plasma samples, stratified by the time of each prospectively collected sample before the date of TB disease diagnosis.

FIG. 3 shows Receiver Operator Characteristics Area under the curve (ROC-AUC) analysis of the 3PR signature for all GC6 household contact study validation set plasma samples, stratified by the time interval of each prospectively collected sample before the date of TB disease diagnosis.

### DETAILED DESCRIPTION OF THE INVENTION

This invention relates to a method of determining the risk of a human subject with asymptomatic tuberculosis (TB) infection, which may be latent TB infection or apparent latent TB infection and/or after suspected exposure to TB progressing to active tuberculosis disease comprising the steps of quantifying and computationally analysing relative abundances of a collection or panel of two pairs of three protein products (“TB proteomic biomarkers”) derived from a sample obtained from the subject. The invention was developed through a systems biology analysis of the only suitably designed clinical cohorts to date. In the approach, mathematical algorithms were used based upon the analysis of the temporal progression during which human subjects with asymptomatic tuberculosis were ultimately diagnosed with active tuberculosis, as well as the abundances of the protein biomarkers in plasma revealed during that timescale, in order to computationally determine a panel of three TB proteomic biomarkers. The identified 3-protein signature predicts development of tuberculosis disease across a variety of ages (adolescents and adults), infection and exposure statuses, and ethnicities and geographies.

The present invention provides the first validated prognostic 3-protein signature to determine which individuals with an asymptomatic tuberculosis infection should or should not be diagnostically screened for signs and symptoms for diagnosis of active TB disease, or who should or should not be given prophylactic chemotherapy to prevent the onset of active TB disease, and to prevent the spread of TB infection to other individuals.

While the invention will be described in conjunction with certain representative embodiments, it will be understood that the invention is defined by the claims, and is not limited to those embodiments.

One skilled in the art will recognize many methods and materials similar or equivalent to those described herein may be used in the practice of the present invention. The present invention is in no way limited to the methods and materials described.

Unless defined otherwise, technical and scientific terms used herein have the meaning commonly understood by one of ordinary skill in the art to which this invention belongs. Although any methods, devices, and materials similar or equivalent to those described herein can be used in the practice of the invention, certain methods, devices, and materials are described herein.

All publications, published patent documents, and patent applications cited herein are hereby incorporated by reference to the same extent as though each individual publication, published patent document, or patent application was specifically and individually indicated as being incorporated by reference.

As used in this application, including the appended claims, the singular forms “a,” “an,” and “the” include the plural, unless the context clearly dictates otherwise, and may be used interchangeably with “at least one” and “one or more.” Thus, reference to “an aptamer” includes mixtures of aptamers; reference to “a probe” includes mixtures of probes, and the like.

As used herein, the terms “comprises,” “comprising,” “includes,” “including,” “contains,” “containing,” and any variations thereof, are intended to cover a non-exclusive inclusion, such that a process, method, product-by-process, or composition of matter that comprises, includes, or contains an element or list of elements may include other elements not expressly listed.

The present application includes biomarkers, methods, devices, reagents, systems, and kits for detecting, characterizing, monitoring progression, and/or monitoring treatment of TB infection and/or TB disease.

As used herein, “tuberculosis infection” or “TB infection” refers to the infection of an individual with any of a variety of TB disease-causing mycobacteria (e.g., Mycobacterium tuberculosis). TB infection encompasses both “latent TB infection” (non-transmissible and without symptoms) and “active TB infection” (transmissible and symptomatic). Observable signs of active TB infection include, but are not limited to, chronic cough with blood-tinged sputum, fever, night sweats, and weight loss. As used herein, “individual” and “subject” and “patient” are used interchangeably to refer to a test subject or patient. The individual can be a mammal or a non-mammal. In various embodiments, the individual is a mammal. A mammalian individual can be a human or non-human. In various embodiments, the individual is a human. A “non-infected” individual is one which has not been infected with a TB disease-causing mycobacterium (e.g., Mycobacterium tuberculosis), does not have either latent TB infection or active TB disease, and/or for whom TB infection is not detectable by conventional diagnostic methods. “Active tuberculosis disease” means a diagnosis of tuberculosis disease based on a positive microbiology laboratory test using sputum or another respiratory specimen that confirms detection of acid-fast bacilli, including XpertTB-RIF®, smear microscopy or sputum culture test.

As used herein, a “subject at risk of TB disease” refers to a subject with or exposed to one or more risk factors for TB disease. Such risk factors include HIV infection, poverty, geographic location, chronic lung disease, poverty, diabetes, genetic susceptibility, imprisonment, etc.

As used herein, the term “progressor” means an asymptomatic, otherwise healthy individual who does not have definite or suspected TB disease, despite other possible infections or diseases, who developed definite TB disease during follow-up in either the ACS or GC6 studies.

The terms “sensitivity” and “specificity” are used herein with respect to the ability to correctly classify an individual, based the biomarker levels detected in a biological sample. “Sensitivity” indicates the performance of the biomarkers with respect to correctly classifying individuals as, for example at risk (e.g., high risk or likely) of transitioning from latent TB infection to active TB disease. “Specificity” indicates the performance of the biomarkers with respect to correctly classifying individuals who have latent TB infection and are not at risk (e.g., low risk) of transitioning from latent TB infection to active TB disease. For example, 85% specificity and 90% sensitivity for a panel of markers used to test a set of control samples (such as samples from individuals with latent TB infections that did not advance to active TB disease) and test samples (such as samples from TB-infected individuals that developed active TB disease) indicates that 85% of the control samples were correctly classified as control samples by the panel, and 90% of the test samples were correctly classified as test samples by the panel.

The area-under-the-curve (AUC) value is derived from receiver operating characteristic (ROC) analyses, which are exemplified herein. The ROC curve is the plot of the true positive rate (sensitivity) of a test against the false positive rate (1—specificity) of the test. The term “area under the curve” or “AUC” refers to the area under the curve of a receiver operating characteristic (ROC) curve, both of which are well known in the art. AUC measures are useful for comparing the accuracy of a classifier across the complete data range. Classifiers with a greater AUC have a greater capacity to classify unknowns correctly between two groups of interest (e.g., low-risk vs. high risk individuals). ROC curves are useful for plotting the performance of a particular feature (e.g., the biomarkers described herein and/or any item of additional biomedical information) in distinguishing between two populations (e.g., cases in which subjects transitioned from latent to active TB vs. controls in which TB infection remained latent). Typically, the feature data across the entire population (e.g., all tested subject) are sorted in ascending order based on the value of a single feature. Then, for each value for that feature, the true positive and false positive rates for the data are calculated. The true positive rate is determined by counting the number of cases above the value for that feature and then dividing by the total number of cases. The false positive rate is determined by counting the number of controls above the value for that feature and then dividing by the total number of controls. Although this definition refers to scenarios in which a feature is elevated in cases compared to controls, this definition also applies to scenarios in which a feature is lower in cases compared to the controls (in such a scenario, samples below the value for that feature would be counted). ROC curves can be generated for a single feature as well as for other single outputs, for example, a combination of two or more features can be mathematically combined (e.g., added, subtracted, multiplied, etc.) to provide a single sum value, and this single sum value can be plotted in a ROC curve. Additionally, any combination of multiple features, in which the combination derives a single output value, can be plotted in a ROC curve.

“Biological sample”, “sample”, and “test sample” are used interchangeably herein to refer to any material, biological fluid, tissue, or cell obtained or otherwise derived from an individual. This includes blood (including whole blood, leukocytes, peripheral blood mononuclear cells, buffy coat, plasma, and serum), sputum, tears, mucus, nasal washes, nasal aspirate, breath, urine, semen, saliva, peritoneal washings, ascites, cystic fluid, meningeal fluid, amniotic fluid, glandular fluid, lymph fluid, nipple aspirate, bronchial aspirate (e.g., bronchoalveolar lavage), bronchial brushing, synovial fluid, joint aspirate, organ secretions, cells, a cellular extract, pleural fluid, pericardial fluid and cerebrospinal fluid. This also includes experimentally separated fractions of all of the preceding. For example, a blood sample can be fractionated into serum, plasma, or into fractions containing particular types of blood cells, such as red blood cells or white blood cells (leukocytes). For example, a sample can be a combination of samples from an individual, such as a combination of a tissue and fluid sample. The term “biological sample” also includes materials containing homogenized solid material, such as from a stool sample, a tissue sample, or a tissue biopsy, for example. The term “biological sample” also includes materials derived from a tissue culture or a cell culture. Any suitable methods for obtaining a biological sample can be employed; exemplary methods include, e.g., phlebotomy, swab (e.g., buccal swab), and a fine needle aspirate biopsy procedure. Exemplary tissues susceptible to fine needle aspiration include lymph node, lung, lung washes, BAL (bronchoalveolar lavage), thyroid, breast, pancreas, and liver. Samples can also be collected, e.g., by micro dissection (e.g., laser capture micro dissection (LCM) or laser micro dissection (LMD)), bladder wash, smear (e.g., a PAP smear), or ductal lavage. A “biological sample” obtained or derived from an individual includes any such sample that has been processed in any suitable manner after being obtained from the individual.

Further, in some embodiments, a biological sample may be derived by taking biological samples from a number of individuals and pooling them, or pooling an aliquot of each individual's biological sample. The pooled sample may be treated as described herein for a sample from a single individual, and, for example, if high-risk TB infection is detected in the pooled sample, then each individual biological sample can be re-tested to identify the individual(s) with latent TB infection that is likely to transition into active TB disease within 30 days, 45 days, 60 days, 90 days, 120 days, 180 days, 270 days, 360 days, 450 days, or 540 days.

“Target”, “target molecule”, and “analyte” are used interchangeably herein to refer to any molecule of interest that may be present in a biological sample. A “molecule of interest” includes any minor variation of a particular molecule, such as, in the case of a protein, for example, minor variations in amino acid sequence, disulfide bond formation, glycosylation, lipidation, acetylation, phosphorylation, or any other manipulation or modification, such as conjugation with a labeling component, which does not substantially alter the identity of the molecule. A “target molecule”, “target”, or “analyte” refers to a set of copies of one type or species of molecule or multi-molecular structure. “Target molecules”, “targets”, and “analytes” refer to more than one type or species of molecule or multi-molecular structure. Exemplary target molecules include proteins, polypeptides, nucleic acids, carbohydrates, lipids, polysaccharides, glycoproteins, hormones, receptors, antigens, antibodies, affybodies, antibody mimics, viruses, pathogens, toxic substances, substrates, metabolites, transition state analogs, cofactors, inhibitors, drugs, dyes, nutrients, growth factors, cells, tissues, and any fragment or portion of any of the foregoing. In some embodiments, a target molecule is a protein, in which case the target molecule may be referred to as a “target protein.”

As used herein, a “capture agent' or “capture reagent” refers to a molecule that is capable of binding specifically to a biomarker. A “target protein capture reagent” refers to a molecule that is capable of binding specifically to a target protein. Nonlimiting exemplary capture reagents include aptamers, antibodies, adnectins, ankyrins, other antibody mimetics and other protein scaffolds, autoantibodies, chimeras, small molecules, nucleic acids, lectins, ligand-binding receptors, imprinted polymers, avimers, peptidomimetics, hormone receptors, cytokine receptors, synthetic receptors, and modifications and fragments of any of the aforementioned capture reagents. Preferably, a capture reagent is selected from an aptamer and an antibody.

The term “antibody” refers to full-length antibodies of any species and fragments and derivatives of such antibodies, including Fab fragments, F(ab′)₂ fragments, single chain antibodies, Fv fragments, and single chain Fv fragments. The term “antibody” also refers to synthetically-derived antibodies, such as phage display-derived antibodies and fragments, affybodies, nanobodies, etc.

As used herein, “marker” and “biomarker” are used interchangeably to refer to a target molecule that indicates or is a sign of a normal or abnormal process in an individual or of a disease or other condition in an individual. More specifically, a “marker” or “biomarker” is an anatomic, physiologic, biochemical, or molecular parameter associated with the presence of a specific physiological state or process, whether normal or abnormal, and, if abnormal, whether chronic or acute. Biomarkers are detectable and measurable by a variety of methods including laboratory assays and medical imaging.

As used herein, “biomarker level” and “level” refer to a measurement that is made using any analytical method for detecting the biomarker in a biological sample and that indicates the presence, absence, absolute amount or concentration, relative amount or concentration, titer, a level, an expression level, a ratio of measured levels, or the like, of, for, or corresponding to the biomarker in the biological sample. The exact nature of the “level” depends on the specific design and components of the particular analytical method employed to detect the biomarker.

A “control level” of a target molecule refers to the level of the target molecule in the same sample type from an individual that does not exhibit the characteristic being assayed for (e.g., TB infection, risk of transition from latent TB infection to active TB disease, etc.).

A “threshold level” of a target molecule refers to the level beyond which (e.g., above or below, depending upon the biomarker) is indicative of or diagnostic for a particular infection, disease, condition, or characteristic thereof. For example, a threshold level of for the likelihood of latent TB infection transitioning into active TB disease is a level of a target molecule beyond which (e.g., above or below, depending upon the biomarker) is indicative of a latnet TB infection that is likely to transition into active TB disease within 30 days, 45 days, 60 days, 90 days, 120 days, 180 days, 270 days, 360 days, 450 days, or 540 days. A “threshold level” of a target molecule need not be determined each time the present methods are carried out, and may be a previously determined level that is used as a reference or threshold to determine whether the level in a particular sample is higher or lower than a normal level. In some embodiments, a subject with a biomarker level beyond (e.g., above or below, depending upon the biomarker) a threshold level has a statistically significant likelihood (e.g., 80% confidence, 85% confidence, 90% confidence, 95% confidence, 98% confidence, 99% confidence, 99.9% confidence, etc.) of having a latent TB infection transition into active TB disease.

“Diagnose”, “diagnosing”, “diagnosis”, and variations thereof refer to the detection, determination, or recognition of a health status or condition of an individual on the basis of one or more signs, symptoms, data, or other information pertaining to that individual. The health status of an individual can be diagnosed as healthy/normal (e.g., a diagnosis of the absence of a disease or condition), diagnosed as ill/abnormal (e.g., a diagnosis of the presence, or an assessment of the characteristics, of a disease or condition), and/or high-risk/low-risk (e.g., of developing a disease or condition, of transitioning from a latent infection to an active disease state). The terms “diagnose”, “diagnosing”, “diagnosis”, etc., encompass, with respect to a particular disease or condition: the initial detection of the disease; the characterization or classification of the disease; the characterization of likelihood of advancement of the disease (e.g., from latent to active); the detection of the progression, remission, or recurrence of the disease; and/or the detection of disease response after the administration of a treatment or therapy to the individual.

“Prognose”, “prognosing”, “prognosis”, “prognostic”, and variations thereof refer to the prediction of a future course of a disease or condition in an individual who has the disease or condition (e.g., predicting patient survival, predicting likelihood of transition from latent infection to active disease, etc.), and such terms encompass the evaluation of disease response after the administration of a treatment or therapy to the individual.

“Evaluate”, “evaluating”, “evaluation”, and variations thereof encompass both “diagnose” and “prognose” and also encompass determinations or predictions about the future course of a disease or condition in an individual who does not have the disease as well as determinations or predictions regarding the likelihood that a disease or condition will recur in an individual who apparently has been cured of the disease. The term “evaluate” also encompasses assessing an individual's response to a therapy, such as, for example, predicting whether an individual is likely to respond favorably to a therapeutic agent or is unlikely to respond to a therapeutic agent (or will experience toxic or other undesirable side effects, for example), selecting a therapeutic agent for administration to an individual, or monitoring or determining an individual's response to a therapy that has been administered to the individual. Thus, “evaluating” TB can include, for example, any of the following: diagnosing a subject with TB infection, diagnosing a subject as suffering from TB disease, determining a subject should undergo further testing (e.g., chest x-ray for TB); prognosing the future course of TB infection/disease in an individual; prognosing a the likelihood of TB transitioning from latent to active; determining whether a TB treatment being administered is effective in the individual; or determining or predicting an individual's response to a TB treatment; or selecting a TB treatment to administer to an individual based upon a determination of the biomarker levels derived from the individual's biological sample.

As used herein, “detecting” or “determining” with respect to a biomarker level includes the use of both the instrument used to observe and record a signal corresponding to a biomarker level and the material/s required to generate that signal. In various embodiments, the level is detected using any suitable method, including fluorescence, chemiluminescence, surface plasmon resonance, surface acoustic waves, mass spectrometry, infrared spectroscopy, Raman spectroscopy, atomic force microscopy, scanning tunneling microscopy, electrochemical detection methods, nuclear magnetic resonance, quantum dots, and the like.

“Solid support” refers herein to any substrate having a surface to which molecules may be attached, directly or indirectly, through either covalent or non-covalent bonds. A “solid support” can have a variety of physical formats, which can include, for example, a membrane; a chip (e.g., a protein chip); a slide (e.g., a glass slide or coverslip); a column; a hollow, solid, semi-solid, pore- or cavity-containing particle, such as, for example, a bead; a gel; a fiber, including a fiber optic material; a matrix; and a sample receptacle. Exemplary sample receptacles include sample wells, tubes, capillaries, vials, and any other vessel, groove or indentation capable of holding a sample. A sample receptacle can be contained on a multi-sample platform, such as a microtiter plate, slide, microfluidics device, and the like. A support can be composed of a natural or synthetic material, an organic or inorganic material. The composition of the solid support on which capture reagents are attached generally depends on the method of attachment (e.g., covalent attachment). Other exemplary receptacles include microdroplets and microfluidic controlled or bulk oil/aqueous emulsions within which assays and related manipulations can occur. Suitable solid supports include, for example, plastics, resins, polysaccharides, silica or silica-based materials, functionalized glass, modified silicon, carbon, metals, inorganic glasses, membranes, nylon, natural fibers (such as, for example, silk, wool and cotton), polymers, and the like. The material composing the solid support can include reactive groups such as, for example, carboxy, amino, or hydroxyl groups, which are used for attachment of the capture reagents. Polymeric solid supports can include, e.g., polystyrene, polyethylene glycol tetraphthalate, polyvinyl acetate, polyvinyl chloride, polyvinyl pyrrolidone, polyacrylonitrile, polymethyl methacrylate, polytetrafluoroethylene, butyl rubber, styrenebutadiene rubber, natural rubber, polyethylene, polypropylene, (poly)tetrafluoroethylene, (poly)vinylidenefluoride, polycarbonate, and polymethylpentene. Suitable solid support particles that can be used include, e.g., encoded particles, such as Luminex®-type encoded particles, magnetic particles, and glass particles.

The molecular techniques referenced herein, including protein extraction and purification and protein capture and quantification, are all standard methods known to those skilled in the art. Many reference sources are available, including but not limited to: http://www.qiagen.com/resources/molecular-biology-methods/, Methods in Molecular Biology, Ed. J. M. Walker, HumanaPress, ISSN: 1064-3745, Molecular Cloning: A Laboratory Manual by Michael R Green and Joseph Sambrook 2012, Cold Spring Harbour Laboratory Press, ISBN: 978-1-936113-42-2, Molecular cloning: a laboratory manual by Tom Maniatis, E. F. Fritsch, Joseph Sambrook 1982, Cold Spring Harbour Laboratory Press and others known to those skilled in the art.

Detection and Determination of Biomarkers and Biomarker Levels

A biomarker level for the biomarkers described herein can be detected using any of a variety of known analytical methods. Preferably a biomarker level is detected using a capture reagent. For example, the capture reagent can be exposed to the biomarker in solution or can be exposed to the biomarker while the capture reagent is immobilized on a solid support. The capture reagent may contain a feature that is reactive with a secondary feature on a solid support. In this case the capture reagent can be exposed to the biomarker in solution, and then the feature on the capture reagent can be used in conjunction with the secondary feature on the solid support to immobilize the biomarker on the solid support. The capture reagent is selected based on the type of analysis to be conducted. Capture reagents include but are not limited to aptamers, antibodies, adnectins, ankyrins, other antibody mimetics and other protein scaffolds, autoantibodies, chimeras, small molecules, F(ab′)₂ fragments, single chain antibody fragments, Fv fragments, single chain Fv fragments, nucleic acids, lectins, ligand-binding receptors, affybodies, nanobodies, imprinted polymers, avimers, peptidomimetics, hormone receptors, cytokine receptors, and synthetic receptors, and modifications and fragments of these.

Biomarker presence or level may be detected using a biomarker/capture reagent complex. For example, the biomarker presence or level may be derived from the biomarker/capture reagent complex and detected indirectly, such as, for example, as a result of a reaction that is subsequent to the biomarker/capture reagent interaction, but is dependent on the formation of the biomarker/capture reagent complex.

The biomarker presence or level may be detected directly from the biomarker in a biological sample.

The biomarkers may be detected using a multiplexed format that allows for the simultaneous detection of two or more biomarkers in a biological sample. For example, capture reagents may be immobilized, directly or indirectly, covalently or non-covalently, in discrete locations on a solid support. A multiplexed format may use discrete solid supports where each solid support has a unique capture reagent associated with that solid support, such as, for example quantum dots. An individual device may be used for the detection of each one of multiple biomarkers to be detected in a biological sample. Individual devices can be configured to permit each biomarker in the biological sample to be processed simultaneously. For example, a microtiter plate can be used such that each well in the plate is used to analyze one or more of multiple biomarkers to be detected in a biological sample.

A fluorescent tag may be used to label a component of the biomarker/capture reagent complex to enable the detection of the biomarker level. The fluorescent label can be conjugated to a capture reagent specific to any of the biomarkers described herein using known techniques, and the fluorescent label can then be used to detect the corresponding biomarker level. Suitable fluorescent labels include rare earth chelates, fluorescein and its derivatives, rhodamine and its derivatives, dansyl, allophycocyanin, PBXL-3, Qdot 605, Lissamine, phycoerythrin, Texas Red, and other such compounds.

The fluorescent label may be a fluorescent dye molecule. For example, the fluorescent dye molecule may include at least one substituted indolium ring system in which the substituent on the 3-carbon of the indolium ring contains a chemically reactive group or a conjugated substance. The dye molecule may include an AlexFluor molecule, such as, for example, AlexaFluor 488, AlexaFluor 532, AlexaFluor 647, AlexaFluor 680, or AlexaFluor 700. The dye molecule may include a first type and a second type of dye molecule, such as, e.g., two different AlexaFluor molecules. The dye molecule may include a first type and a second type of dye molecule, and the two dye molecules may have different emission spectra.

Fluorescence can be measured with a variety of instrumentation compatible with a wide range of assay formats. For example, spectrofluorimeters have been designed to analyze microtiter plates, microscope slides, printed arrays, cuvettes, etc. See Principles of Fluorescence Spectroscopy, by J. R. Lakowicz, Springer Science+Business Media, Inc., 2004. See Bioluminescence & Chemiluminescence: Progress & Current Applications; Philip E. Stanley and Larry J. Kricka editors, World Scientific Publishing Company, January 2002.

A chemiluminescence tag may optionally be used to label a component of the biomarker/capture complex to enable the detection of a biomarker level. Suitable chemiluminescent materials include any of oxalyl chloride, Rodamin 6G, Ru(bipy)32+, TMAE (tetrakis(dimethylamino)ethylene), Pyrogallol (1,2,3-trihydroxibenzene), Lucigenin, peroxyoxalates, Aryl oxalates, Acridinium esters, dioxetanes, and others.

The detection method may include an enzyme/substrate combination that generates a detectable signal that corresponds to the biomarker level. Generally, the enzyme catalyzes a chemical alteration of the chromogenic substrate which can be measured using various techniques, including spectrophotometry, fluorescence, and chemiluminescence. Suitable enzymes include, for example, luciferases, luciferin, malate dehydrogenase, urease, horseradish peroxidase (HRPO), alkaline phosphatase, beta-galactosidase, glucoamylase, lysozyme, glucose oxidase, galactose oxidase, and glucose-6-phosphate dehydrogenase, uricase, xanthine oxidase, lactoperoxidase, microperoxidase, and the like.

The detection method may be a combination of fluorescence, chemiluminescence, radionuclide or enzyme/substrate combinations that generate a measurable signal. Multimodal signaling may have unique and advantageous characteristics in certain biomarker assay formats.

The biomarker levels for the biomarkers described herein may be detected using any analytical methods including, singleplex aptamer assays, multiplexed aptamer assays, singleplex or multiplexed immunoassays, mass spectrometric analysis, histological/cytological methods, etc.

Determination of Biomarker Levels Using Aptamers

As used herein, an “aptamer” refers to a nucleic acid that has a specific binding affinity for a target molecule. It is recognized that affinity interactions are a matter of degree; however, in this context, the “specific binding affinity” of an aptamer for its target means that the aptamer binds to its target generally with a much higher degree of affinity than it binds to other components in a test sample. An “aptamer” is a set of copies of one type or species of nucleic acid molecule that has a particular nucleotide sequence. An aptamer can include any suitable number of nucleotides, including any number of chemically modified nucleotides. “Aptamers” refers to more than one such set of molecules. Different aptamers can have either the same or different numbers of nucleotides. Aptamers can be DNA or RNA or chemically modified nucleic acids and can be single stranded, double stranded, or contain double stranded regions, and can include higher ordered structures. An aptamer can also be a photoaptamer, where a photoreactive or chemically reactive functional group is included in the aptamer to allow it to be covalently linked to its corresponding target. Any of the aptamer methods disclosed herein can include the use of two or more aptamers that specifically bind the same target molecule. An aptamer may include a tag. If an aptamer includes a tag, all copies of the aptamer need not have the same tag. Moreover, if different aptamers each include a tag, these different aptamers can have either the same tag or a different tag.

An aptamer can be identified using any known method, including the SELEX process. Once identified, an aptamer can be prepared or synthesized in accordance with any known method, including chemical synthetic methods and enzymatic synthetic methods.

The terms “SELEX” and “SELEX process” are used interchangeably herein to refer generally to a combination of (1) the selection of aptamers that interact with a target molecule in a desirable manner, for example binding with high affinity to a protein, with (2) the amplification of those selected nucleic acids. The SELEX process can be used to identify aptamers with high affinity to a specific target or biomarker.

SELEX generally includes preparing a candidate mixture of nucleic acids, binding of the candidate mixture to the desired target molecule to form an affinity complex, separating the affinity complexes from the unbound candidate nucleic acids, separating and isolating the nucleic acid from the affinity complex, purifying the nucleic acid, and identifying a specific aptamer sequence. The process may include multiple rounds to further refine the affinity of the selected aptamer. The process can include amplification steps at one or more points in the process. See, e.g., U.S. Pat. No. 5,475,096, entitled “Nucleic Acid Ligands”. The SELEX process can be used to generate an aptamer that covalently binds its target as well as an aptamer that non-covalently binds its target. See, e.g., U.S. Pat. No. 5,705,337 entitled “Systematic Evolution of Nucleic Acid Ligands by Exponential Enrichment: Chemi-SELEX.”

The SELEX process can be used to identify high-affinity aptamers containing modified nucleotides that confer improved characteristics on the aptamer, such as, for example, improved in vivo stability or improved delivery characteristics. Examples of such modifications include chemical substitutions at the ribose and/or phosphate and/or base positions. SELEX process-identified aptamers containing modified nucleotides are described in U.S. Pat. No. 5,660,985, entitled “High Affinity Nucleic Acid Ligands Containing Modified Nucleotides”, which describes oligonucleotides containing nucleotide derivatives chemically modified at the 5′- and 2′-positions of pyrimidines. U.S. Pat. No. 5,580,737, see supra, describes highly specific aptamers containing one or more nucleotides modified with 2′-amino (2′-NH₂), 2′-fluoro (2′-F), and/or 2′-O-methyl (2′-OMe). See also, U.S. Patent Application Publication No. 2009/0098549, entitled “SELEX and PHOTOSELEX”, which describes nucleic acid libraries having expanded physical and chemical properties and their use in SELEX and photoSELEX.

SELEX can also be used to identify aptamers that have desirable off-rate characteristics. See U.S. Publication No. US 2009/0004667, entitled “Method for Generating Aptamers with Improved Off-Rates”, which describes improved SELEX methods for generating aptamers that can bind to target molecules. Methods for producing aptamers and photoaptamers having slower rates of dissociation from their respective target molecules are described. The methods involve contacting the candidate mixture with the target molecule, allowing the formation of nucleic acid-target complexes to occur, and performing a slow off-rate enrichment process wherein nucleic acid-target complexes with fast dissociation rates will dissociate and not reform, while complexes with slow dissociation rates will remain intact. Additionally, the methods include the use of modified nucleotides in the production of candidate nucleic acid mixtures to generate aptamers with improved off-rate performance. An aptamer may comprise at least one nucleotide with a modification, such as a base modification. An aptamer may comprise at least one nucleotide with a hydrophobic modification, such as a hydrophobic base modification, allowing for hydrophobic contacts with a target protein. Such hydrophobic contacts, contribute to greater affinity and/or slower off-rate binding by the aptamer. An aptamer may comprise at least two, at least three, at least four, at least five, at least six, at least seven, at least eight, at least nine, or at least 10 nucleotides with hydrophobic modifications, where each hydrophobic modification may be the same or different from the others.

A slow off-rate aptamer (including an aptamers comprising at least one nucleotide with a hydrophobic modification) may have an off-rate (t½) of ≥30 minutes, ≥60 minutes, ≥90 minutes, ≥120 minutes, ≥150 minutes, ≥180 minutes, ≥210 minutes, or ≥240 minutes.

As used herein, a “SOMAmer” or “Slow Off-Rate Aptamer” refers to an aptamer having improved off-rate characteristics. Slow off-rate aptamers can be generated using the modified SELEX methods described in U.S. Publication No. 20090004667; herein incorporated by reference in its entirety. The methods disclosed herein are in no way limited to slow off-rate aptamers, however, use of the slow off-rate process described in U.S. Pat. No. 7,964,356 and U.S. Publication No. 2012/0115752 (herein incorporated by reference in their entireties), may provide improved results.

An assay may employ aptamers that include photoreactive functional groups that enable the aptamers to covalently bind or “photocrosslink” their target molecules. See, e.g., U.S. Pat. No. 6,544,776 entitled “Nucleic Acid Ligand Diagnostic Biochip”. These photoreactive aptamers are also referred to as photoaptamers. See, e.g., U.S. Pat. Nos. 5,763,177, 6,001,577, and 6,291,184, each of which is entitled “Systematic Evolution of Nucleic Acid Ligands by Exponential Enrichment: Photoselection of Nucleic Acid Ligands and Solution SELEX”; see also, e.g., U.S. Pat. No. 6,458,539, entitled “Photoselection of Nucleic Acid Ligands”. After the microarray is contacted with the sample and the photoaptamers have had an opportunity to bind to their target molecules, the photoaptamers are photoactivated, and the solid support is washed to remove any non-specifically bound molecules. Harsh wash conditions may be used, since target molecules that are bound to the photoaptamers are generally not removed, due to the covalent bonds created by the photoactivated functional group(s) on the photoaptamers. In this manner, the assay enables the detection of a biomarker level corresponding to a biomarker in the test sample.

In some assay formats, the aptamers are immobilized on the solid support prior to being contacted with the sample. Under certain circumstances, however, immobilization of the aptamers prior to contact with the sample may not provide an optimal assay. For example, pre-immobilization of the aptamers may result in inefficient mixing of the aptamers with the target molecules on the surface of the solid support, perhaps leading to lengthy reaction times and, therefore, extended incubation periods to permit efficient binding of the aptamers to their target molecules. Further, when photoaptamers are employed in the assay and depending upon the material utilized as a solid support, the solid support may tend to scatter or absorb the light used to effect the formation of covalent bonds between the photoaptamers and their target molecules. Moreover, depending upon the method employed, detection of target molecules bound to their aptamers can be subject to imprecision, since the surface of the solid support may also be exposed to and affected by any labeling agents that are used. Finally, immobilization of the aptamers on the solid support generally involves an aptamer-preparation step (i.e., the immobilization) prior to exposure of the aptamers to the sample, and this preparation step may affect the activity or functionality of the aptamers.

Aptamer assays that permit an aptamer to capture its target in solution and then employ separation steps that are designed to remove specific components of the aptamer-target mixture prior to detection have also been described (see U.S. Publication No. 2009/0042206, entitled “Multiplexed Analyses of Test Samples”). The described aptamer assay methods enable the detection and quantification of a non-nucleic acid target (e.g., a protein target) in a test sample by detecting and quantifying a nucleic acid (i.e., an aptamer). The described methods create a nucleic acid surrogate (i.e., the aptamer) for detecting and quantifying a non-nucleic acid target, thus allowing the wide variety of nucleic acid technologies, including amplification, to be applied to a broader range of desired targets, including protein targets.

Aptamers can be constructed to facilitate the separation of the assay components from an aptamer biomarker complex (or photoaptamer biomarker covalent complex) and permit isolation of the aptamer for detection and/or quantification. In one embodiment, these constructs can include a cleavable or releasable element within the aptamer sequence. In other embodiments, additional functionality can be introduced into the aptamer, for example, a labeled or detectable component, a spacer component, or a specific binding tag or immobilization element. For example, the aptamer can include a tag connected to the aptamer via a cleavable moiety, a label, a spacer component separating the label, and the cleavable moiety. In one embodiment, a cleavable element is a photocleavable linker. The photocleavable linker can be attached to a biotin moiety and a spacer section, can include an NHS group for derivatization of amines, and can be used to introduce a biotin group to an aptamer, thereby allowing for the release of the aptamer later in an assay method.

Homogenous assays, done with all assay components in solution, do not require separation of sample and reagents prior to the detection of signal. These methods are rapid and easy to use. These methods generate signal based on a molecular capture or binding reagent that reacts with its specific target.

A method for signal generation takes advantage of anisotropy signal change due to the interaction of a fluorophore-labeled capture reagent with its specific biomarker target. When the labeled capture reacts with its target, the increased molecular weight causes the rotational motion of the fluorophore attached to the complex to become much slower changing the anisotropy value. By monitoring the anisotropy change, binding events may be used to quantitatively measure the biomarkers in solutions. Other methods include fluorescence polarization assays, molecular beacon methods, time resolved fluorescence quenching, chemiluminescence, fluorescence resonance energy transfer, and the like.

An exemplary solution-based aptamer assay that can be used to detect a biomarker level in a biological sample includes the following: (a) preparing a mixture by contacting the biological sample with an aptamer that includes a first tag and has a specific affinity for the biomarker, wherein an aptamer affinity complex is formed when the biomarker is present in the sample; (b) exposing the mixture to a first solid support including a first capture element, and allowing the first tag to associate with the first capture element; (c) removing any components of the mixture not associated with the first solid support; (d) attaching a second tag to the biomarker component of the aptamer affinity complex; (e) releasing the aptamer affinity complex from the first solid support; (f) exposing the released aptamer affinity complex to a second solid support that includes a second capture element and allowing the second tag to associate with the second capture element; (g) removing any non-complexed aptamer from the mixture by partitioning the non-complexed aptamer from the aptamer affinity complex; (h) eluting the aptamer from the solid support; and (i) detecting the biomarker by detecting the aptamer component of the aptamer affinity complex.

A non-limiting exemplary method of detecting biomarkers in a biological sample using aptamers is described, for example, in Kraemer et al., 2011, PLoS One 6(10): e26332; herein incorporated by reference in its entirety.

Determination of Biomarker Levels Using Immunoassays

Immunoassay methods are based on the reaction of an antibody to its corresponding target or analyte and can detect the analyte in a sample depending on the specific assay format. To improve specificity and sensitivity of an assay method based on immuno-reactivity, monoclonal antibodies and fragments thereof are often used because of their specific epitope recognition. Polyclonal antibodies have also been successfully used in various immunoassays because of their increased affinity for the target as compared to monoclonal antibodies. Immunoassays have been designed for use with a wide range of biological sample matrices. Immunoassay formats have been designed to provide qualitative, semi-quantitative, and quantitative results.

Quantitative results are generated through the use of a standard curve created with known concentrations of the specific analyte to be detected. The response or signal from an unknown sample is plotted onto the standard curve, and a quantity or level corresponding to the target in the unknown sample is established.

Numerous immunoassay formats have been designed. ELISA or EIA can be quantitative for the detection of an analyte. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I125) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or for quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 386 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

Detection of Biomarkers Using In Vivo Molecular Imaging Technologies

In some embodiments, a biomarker described herein may be used in molecular imaging tests. For example, an imaging agent can be coupled to a capture reagent, which can be used to detect the biomarker in vivo.

In vivo imaging technologies provide non-invasive methods for determining the state of a particular disease in the body of an individual. For example, entire portions of the body, or even the entire body, may be viewed as a three dimensional image, thereby providing valuable information concerning morphology and structures in the body. Such technologies may be combined with the detection of the biomarkers described herein to provide information concerning the biomarker in vivo.

The use of in vivo molecular imaging technologies is expanding due to various advances in technology. These advances include the development of new contrast agents or labels, such as radiolabels and/or fluorescent labels, which can provide strong signals within the body; and the development of powerful new imaging technology, which can detect and analyze these signals from outside the body, with sufficient sensitivity and accuracy to provide useful information. The contrast agent can be visualized in an appropriate imaging system, thereby providing an image of the portion or portions of the body in which the contrast agent is located. The contrast agent may be bound to or associated with a capture reagent, such as an aptamer or an antibody, for example, and/or with a peptide or protein, or an oligonucleotide (for example, for the detection of gene expression), or a complex containing any of these with one or more macromolecules and/or other particulate forms.

The contrast agent may also feature a radioactive atom that is useful in imaging. Suitable radioactive atoms include technetium-99m or iodine-123 for scintigraphic studies. Other readily detectable moieties include, for example, spin labels for magnetic resonance imaging (MRI) such as, for example, iodine-123 again, iodine-131, indium-111, fluorine-19, carbon-13, nitrogen-15, oxygen-17, gadolinium, manganese or iron. Such labels are well known in the art and could easily be selected by one of ordinary skill in the art.

Standard imaging techniques include but are not limited to magnetic resonance imaging, computed tomography scanning, positron emission tomography (PET), single photon emission computed tomography (SPECT), and the like. For diagnostic in vivo imaging, the type of detection instrument available is a major factor in selecting a given contrast agent, such as a given radionuclide and the particular biomarker that it is used to target (protein, mRNA, and the like). The radionuclide chosen typically has a type of decay that is detectable by a given type of instrument. Also, when selecting a radionuclide for in vivo diagnosis, its half-life should be long enough to enable detection at the time of maximum uptake by the target tissue but short enough that deleterious radiation of the host is minimized.

Exemplary imaging techniques include but are not limited to PET and SPECT, which are imaging techniques in which a radionuclide is synthetically or locally administered to an individual. The subsequent uptake of the radiotracer is measured over time and used to obtain information about the targeted tissue and the biomarker. Because of the high-energy (gamma-ray) emissions of the specific isotopes employed and the sensitivity and sophistication of the instruments used to detect them, the two-dimensional distribution of radioactivity may be inferred from outside of the body.

Commonly used positron-emitting nuclides in PET include, for example, carbon-11, nitrogen-13, oxygen-15, and fluorine-18. Isotopes that decay by electron capture and/or gamma-emission are used in SPECT and include, for example iodine-123 and technetium-99m. An exemplary method for labeling amino acids with technetium-99m is the reduction of pertechnetate ion in the presence of a chelating precursor to form the labile technetium-99m-precursor complex, which, in turn, reacts with the metal binding group of a bifunctionally modified chemotactic peptide to form a technetium-99m-chemotactic peptide conjugate.

Antibodies are frequently used for such in vivo imaging diagnostic methods. The preparation and use of antibodies for in vivo diagnosis is well known in the art. Similarly, aptamers may be used for such in vivo imaging diagnostic methods. For example, an aptamer that was used to identify a particular biomarker described herein may be appropriately labeled and injected into an individual to detect the biomarker in vivo. The label used will be selected in accordance with the imaging modality to be used, as previously described. Aptamer-directed imaging agents could have unique and advantageous characteristics relating to tissue penetration, tissue distribution, kinetics, elimination, potency, and selectivity as compared to other imaging agents.

Such techniques may also optionally be performed with labeled oligonucleotides, for example, for detection of gene expression through imaging with antisense oligonucleotides. These methods are used for in situ hybridization, for example, with fluorescent molecules or radionuclides as the label. Other methods for detection of gene expression include, for example, detection of the activity of a reporter gene.

Another general type of imaging technology is optical imaging, in which fluorescent signals within the subject are detected by an optical device that is external to the subject. These signals may be due to actual fluorescence and/or to bioluminescence. Improvements in the sensitivity of optical detection devices have increased the usefulness of optical imaging for in vivo diagnostic assays.

Other techniques are review, for example, in N. Blow, Nature Methods, 6, 465-469, 2009; herein incorporated by reference in its entirety.

Determination of Biomarkers Using Histology/Cytology Methods

The biomarkers described herein may be detected in a variety of tissue samples using histological or cytological methods. For example, endo- and trans-bronchial biopsies, fine needle aspirates, cutting needles, and core biopsies can be used for histology. Bronchial washing and brushing, pleural aspiration, and sputum, can be used for cyotology. Any of the biomarkers identified herein can be used to stain a specimen as an indication of disease.

One or more capture reagent/s specific to the corresponding biomarker/s may be used in a cytological evaluation of a sample and may include one or more of the following: collecting a cell sample, fixing the cell sample, dehydrating, clearing, immobilizing the cell sample on a microscope slide, permeabilizing the cell sample, treating for analyte retrieval, staining, destaining, washing, blocking, and reacting with one or more capture reagent/s in a buffered solution. In another embodiment, the cell sample is produced from a cell block.

One or more capture reagent/s specific to the corresponding biomarkers may be used in a histological evaluation of a tissue sample and may include one or more of the following: collecting a tissue specimen, fixing the tissue sample, dehydrating, clearing, immobilizing the tissue sample on a microscope slide, permeabilizing the tissue sample, treating for analyte retrieval, staining, destaining, washing, blocking, rehydrating, and reacting with capture reagents in a buffered solution. In another embodiment, fixing and dehydrating are replaced with freezing.

The one or more aptamer/s specific to the corresponding biomarker/s may be reacted with the histological or cytological sample and can serve as the nucleic acid target in a nucleic acid amplification method. Suitable nucleic acid amplification methods include, for example, PCR, q-beta replicase, rolling circle amplification, strand displacement, helicase dependent amplification, loop mediated isothermal amplification, ligase chain reaction, and restriction and circularization aided rolling circle amplification.

The one or more capture reagents specific to the corresponding biomarkers for use in the histological or cytological evaluation may be mixed in a buffered solution that can include any of the following: blocking materials, competitors, detergents, stabilizers, carrier nucleic acid, polyanionic materials, etc.

A “cytology protocol” generally includes sample collection, sample fixation, sample immobilization, and staining. “Cell preparation” can include several processing steps after sample collection, including the use of one or more aptamers for the staining of the prepared cells.

Determination of Biomarker Levels Using Mass Spectrometry Methods

A variety of configurations of mass spectrometers can be used to detect biomarker levels. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al. Anal. Chem. 70:647 R-716R (1998); Kinter and Sherman, New York (2000)).

Protein biomarkers and biomarker levels can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS)N, atmospheric pressure photoionization mass spectrometry (APPI-MS), APPI-MS/MS, and APPI-(MS)N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker levels. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)₂ fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g. diabodies etc) imprinted polymers, avimers, peptidomimetics, peptoids, peptide nucleic acids, threose nucleic acid, a hormone receptor, a cytokine receptor, and synthetic receptors, and modifications and fragments of these.

The foregoing assays enable the detection of biomarker levels that are useful in the methods described herein, where the methods comprise detecting, in a biological sample from an individual, at least one, at least two, at least three, at least four, at least five, at least six, at least seven, at least eight, or at least nine biomarkers selected from the described herein. Thus, while some of the described biomarkers may be useful alone for detecting TB infection, methods are also described herein for the grouping of multiple biomarkers and subsets of the biomarkers to form panels of two or more biomarkers. In accordance with any of the methods described herein, biomarker levels can be detected and classified individually or they can be detected and classified collectively, as for example in a multiplex assay format.

Classification of Biomarkers and Calculation of Disease Scores

A biomarker “signature” for a given diagnostic test typically contains a set of markers, each marker having characteristic levels in the populations of interest. Characteristic levels may refer to the mean or average of the biomarker levels for the individuals in a particular group. A diagnostic method described herein can be used to assign an unknown sample from an individual into one of two groups: for example, active TB or no active TB. The assignment of a sample into one of two or more groups is known as classification, and the procedure used to accomplish this assignment is known as a classifier or a classification method. Classification methods may also be referred to as scoring methods. There are many classification methods that can be used to construct a diagnostic classifier from a set of biomarker levels. In some instances, classification methods are performed using supervised learning techniques in which a data set is collected using samples obtained from individuals within two (or more, for multiple classification states) distinct groups one wishes to distinguish. Since the class (group or population) to which each sample belongs is known in advance for each sample, the classification method can be trained to give the desired classification response. It is also possible to use unsupervised learning techniques to produce a diagnostic classifier.

Common approaches for developing diagnostic classifiers include decision trees; bagging+boosting+forests; rule inference based learning; Parzen Windows; linear models; logistic; neural network methods; unsupervised clustering; K-means; hierarchical ascending/descending; semi-supervised learning; prototype methods; nearest neighbor; kernel density estimation; support vector machines; hidden Markov models; Boltzmann Learning; and classifiers may be combined either simply or in ways which minimize particular objective functions. For a review, see, e.g., Pattern Classification, R. O. Duda, et al., editors, John Wiley & Sons, 2nd edition, 2001; see also, The Elements of Statistical Learning—Data Mining, Inference, and Prediction, T. Hastie, et al., editors, Springer Science+Business Media, LLC, 2nd edition, 2009.

To produce a classifier using supervised learning techniques, a set of samples called training data are obtained. In the context of diagnostic tests, training data includes samples from the distinct groups (classes) to which unknown samples will later be assigned. For example, samples collected from individuals in a control population and individuals in a particular disease population can constitute training data to develop a classifier that can classify unknown samples (or, more particularly, the individuals from whom the samples were obtained) as either having the disease or being free from the disease. The development of the classifier from the training data is known as training the classifier. Specific details on classifier training depend on the nature of the supervised learning technique. Training a naïve Bayesian classifier is an example of such a supervised learning technique (see, e.g., Pattern Classification, R. O. Duda, et al., editors, John Wiley & Sons, 2nd edition, 2001; see also, The Elements of Statistical Learning—Data Mining, Inference, and Prediction, T. Hastie, et al., editors, Springer Science+Business Media, LLC, 2nd edition, 2009). Training of a naïve Bayesian classifier is described, e.g., in U.S. Publication Nos: 2012/0101002 and 2012/0077695.

Since typically there are many more potential biomarker levels than samples in a training set, care must be used to avoid over-fitting. Over-fitting occurs when a statistical model describes random error or noise instead of the underlying relationship. Over-fitting can be avoided in a variety of way, including, for example, by limiting the number of markers used in developing the classifier, by assuming that the marker responses are independent of one another, by limiting the complexity of the underlying statistical model employed, and by ensuring that the underlying statistical model conforms to the data.

An illustrative example of the development of a diagnostic test using a set of biomarkers includes the application of a naïve Bayes classifier, a simple probabilistic classifier based on Bayes theorem with strict independent treatment of the biomarkers. Each biomarker is described by a class-dependent probability density function (PDF) for the measured RFU values or log RFU (relative fluorescence units) values in each class. The joint PDFs for the set of markers in one class is assumed to be the product of the individual class-dependent PDFs for each biomarker. Training a naïve Bayes classifier in this context amounts to assigning parameters (“parameterization”) to characterize the class dependent PDFs. Any underlying model for the class-dependent PDFs may be used, but the model should generally conform to the data observed in the training set.

The performance of the naïve Bayes classifier is dependent upon the number and quality of the biomarkers used to construct and train the classifier. A single biomarker will perform in accordance with its KS-distance (Kolmogorov-Smirnov). The addition of subsequent markers with good KS distances (>0.3, for example) will, in general, improve the classification performance if the subsequently added markers are independent of the first marker. Using the sensitivity plus specificity as a classifier score, many high scoring classifiers can be generated with a variation of a greedy algorithm. (A greedy algorithm is any algorithm that follows the problem solving metaheuristic of making the locally optimal choice at each stage with the hope of finding the global optimum.)

Another way to depict classifier performance is through a receiver operating characteristic (ROC), or simply ROC curve or ROC plot. The ROC is a graphical plot of the sensitivity, or true positive rate, vs. false positive rate (1—specificity or 1—true negative rate), for a binary classifier system as its discrimination threshold is varied. The ROC can also be represented equivalently by plotting the fraction of true positives out of the positives (TPR=true positive rate) vs. the fraction of false positives out of the negatives (FPR=false positive rate). Also known as a Relative Operating Characteristic curve, because it is a comparison of two operating characteristics (TPR & FPR) as the criterion changes. The area under the ROC curve (AUC) is commonly used as a summary measure of diagnostic accuracy. It can take values from 0.0 to 1.0. The AUC has an important statistical property: the AUC of a classifier is equivalent to the probability that the classifier will rank a randomly chosen positive instance higher than a randomly chosen negative instance (Fawcett T, 2006. An introduction to ROC analysis. Pattern Recognition Letters .27: 861-874). This is equivalent to the Wilcoxon test of ranks (Hanley, J. A., McNeil, B. J., 1982. The meaning and use of the area under a receiver operating characteristic (ROC) curve. Radiology 143, 29-36.).

Kits

Any combination of the biomarkers described herein can be detected using a suitable kit, such as for use in performing the methods disclosed herein. The biomarkers described herein may be combined in any suitable combination, or may be combined with other markers not described herein. Furthermore, any kit can contain one or more detectable labels as described herein, such as a fluorescent moiety, etc.

In some embodiments, a kit includes (a) one or more capture reagents (such as, for example, at least one aptamer or antibody) for detecting one or more biomarkers in a biological sample, and optionally (b) one or more software or computer program products for predicting whether the individual from whom the biological sample was obtained is TB infected. Alternatively, rather than one or more computer program products, one or more instructions for manually performing the above steps by a human can be provided.

In some embodiments, a kit comprises a solid support, a capture reagent, and a signal generating material. The kit can also include instructions for using the devices and reagents, handling the sample, and analyzing the data. Further the kit may be used with a computer system or software to analyze and report the result of the analysis of the biological sample.

The kits can also contain one or more reagents (e.g., solubilization buffers, detergents, washes, or buffers) for processing a biological sample. Any of the kits described herein can also include, e.g., buffers, blocking agents, mass spectrometry matrix materials, antibody capture agents, positive control samples, negative control samples, software and information such as protocols, guidance and reference data.

For example, a kit can comprise (a) reagents comprising at least one capture reagent for determining the level of one or more biomarkers in a test sample, and optionally (b) one or more algorithms or computer programs for performing the steps of comparing the amount of each biomarker quantified in the test sample to one or more predetermined cutoffs. In some embodiments, an algorithm or computer program assigns a score for each biomarker quantified based on said comparison and, in some embodiments, combines the assigned scores for each biomarker quantified to obtain a total score. Further, in some embodiments, an algorithm or computer program compares the total score with a predetermined score, and uses the comparison to determine, for example, likelihood of latent TB infection advancing into active TB disease. Alternatively, rather than one or more algorithms or computer programs, one or more instructions for manually performing the above steps by a human can be provided.

Methods of Treatment

Following characterization of a subject's TB status (e.g., no infection; latent infection not likely to advance to active TB; latent infection—likely to advance to active TB within 30 days, 45 days, 60 days, 90 days, 120 days, 180 days, 270 days, 360 days, 450 days, or 540 days; active TB disease; etc.), the subject may be treated for TB infection. Medications used to treat latent TB infection include: isoniazid (INH), rifampin (RIF), and rifapentine (RPT). TB disease may be treated by taking several drugs for 6 to 9 months. There are 10 drugs currently approved by the U.S. Food and Drug Administration (FDA) for treating TB. Of the approved drugs, the first-line anti-TB agents that form the core of treatment regimens include: isoniazid (INH), rifampin (RIF), ethambutol (EMB), and pyrazinamide (PZA). Regimens for treating TB disease have an initial phase of 2 months, followed by a choice of several options for the continuation phase of either 4 or 7 months (total of 6 to 9 months for treatment).

Methods of monitoring TB infection/disease and/or treatment of TB infection/disease are provided. For example, the present methods of detecting TB infection are carried out at a time 0. The method may be carried out again at a time 1, and optionally, a time 2, and optionally, a time 3, etc., in order to monitor the progression of TB infection or to monitor the effectiveness of one or more treatments of TB. Time points for detection may be separated by, for example at least 1 day, at least 2 days, at least 4 days, at least 1 week, at least 2 weeks, at least 1 month, at least 2 months, at least 3 months, at least 4 months, at least 6 months, or by 1 year or more. In some embodiments, a treatment regimen is altered based upon the results of monitoring (e.g., upon determining that a first treatment is ineffective).

3-Protein Pair Ratio Signature Score Calculation

The 3 protein signature of risk for TB disease progression was developed using the Pair Ratios algorithm and is a variation on the pairwise approach used to discover the ACS COR signature (Zak et al, 2016). Briefly,
1. The protein concentration for the three proteins C9, C1qTNF3 and CK-MB is quantified.
2. For each of the two pairs of proteins (pair 1 and pair 2), the difference in concentration is computed, generating a log-transformed ratio of expression for pair 1 and pair 2.
3. The measured log-transformed ratios are compared to the ratios provided in the look-up tables for the given pair of proteins listed in Table 1 and Table 2. This is performed by identifying the minimal ratio in column 1 of the resepctive table that is greater than or equal to the measured log-transformed ratio.
4. The corresponding score in the second column of the respective look-up table is then assigned to the measured log-transformed ratio. If the measured log-transformed ratio is greater than all ratios in column 1 of the look-up table, then a score of 1 is assigned to the measured log-transformed ratio. A corresponding score is generated in this way for pair 1 and a corresponding score for pair 2.
5. The average or mean of the scores generated from pair 1 and pair 2 is then computed to generate a final score for the sample. If any assays failed on a sample, the average score over all ratios not including the failed assays is computed. The resulting average is the final score for that sample.

TABLE 1
The look-up table for protein pairs C9 and Cq1TNF3

| Pair ratio | Score |
|---|---|
| −1.04956 | 0 |
| −0.862277 | 0.00172414 |
| −0.828487 | 0.00344828 |
| −0.679094 | 0.00517241 |
| −0.636496 | 0.00689655 |
| −0.580692 | 0.00862069 |
| 0.926295 | 0.0145731 |
| 1.33956 | 0.0162972 |
| 1.52347 | 0.0180213 |
| 1.53077 | 0.0197455 |
| 1.53814 | 0.0214696 |
| 1.57056 | 0.0231938 |
| 1.59333 | 0.0249179 |
| 1.59672 | 0.026642 |
| 1.69222 | 0.0283662 |
| 1.69718 | 0.0300903 |
| 1.71674 | 0.0318144 |
| 1.71854 | 0.0335386 |
| 1.74838 | 0.0352627 |
| 1.75124 | 0.0369869 |
| 1.77395 | 0.038711 |
| 1.78695 | 0.0404351 |
| 1.79164 | 0.0421593 |
| 1.80166 | 0.0438834 |
| 1.80301 | 0.0456076 |
| 1.80568 | 0.0473317 |
| 1.81692 | 0.0490558 |
| 1.832 | 0.05078 |
| 1.84545 | 0.0525041 |
| 1.85866 | 0.0542282 |
| 1.86425 | 0.0559524 |
| 1.87093 | 0.0576765 |
| 1.88394 | 0.0594007 |
| 1.88396 | 0.0611248 |
| 1.89814 | 0.0628489 |
| 1.90167 | 0.0645731 |
| 1.90194 | 0.0662972 |
| 1.90325 | 0.0680213 |
| 1.91347 | 0.0697455 |
| 1.91397 | 0.0714696 |
| 1.91883 | 0.0731938 |
| 1.92232 | 0.0749179 |
| 1.93115 | 0.076642 |
| 1.93245 | 0.0783662 |
| 1.93617 | 0.0843186 |
| 1.93824 | 0.0902709 |
| 1.94038 | 0.0962233 |
| 1.95094 | 0.0979475 |
| 1.95471 | 0.0996716 |
| 1.95947 | 0.101396 |
| 1.95957 | 0.10312 |
| 1.96659 | 0.104844 |
| 1.9796 | 0.106568 |
| 1.97987 | 0.108292 |
| 1.98037 | 0.110016 |
| 1.9855 | 0.111741 |
| 1.9872 | 0.113465 |
| 1.98767 | 0.115189 |
| 1.98837 | 0.116913 |
| 1.99398 | 0.118637 |
| 1.99645 | 0.120361 |
| 1.99792 | 0.122085 |
| 2.00145 | 0.12381 |
| 2.00556 | 0.125534 |
| 2.00681 | 0.127258 |
| 2.00892 | 0.128982 |
| 2.01108 | 0.130706 |
| 2.01367 | 0.13243 |
| 2.02206 | 0.134154 |
| 2.03436 | 0.135878 |
| 2.03534 | 0.137603 |
| 2.0356 | 0.139327 |
| 2.04035 | 0.141051 |
| 2.05575 | 0.142775 |
| 2.05774 | 0.144499 |
| 2.05787 | 0.146223 |
| 2.05857 | 0.147947 |
| 2.05994 | 0.149672 |
| 2.06285 | 0.151396 |
| 2.06536 | 0.15312 |
| 2.06888 | 0.154844 |
| 2.07102 | 0.156568 |
| 2.07249 | 0.158292 |
| 2.07482 | 0.160016 |
| 2.07662 | 0.165969 |
| 2.0788 | 0.171921 |
| 2.07985 | 0.173645 |
| 2.09569 | 0.175369 |
| 2.09848 | 0.177094 |
| 2.09904 | 0.178818 |
| 2.10387 | 0.180542 |
| 2.10636 | 0.182266 |
| 2.1111 | 0.18399 |
| 2.11412 | 0.185714 |
| 2.11565 | 0.187438 |
| 2.11698 | 0.189163 |
| 2.12328 | 0.190887 |
| 2.12711 | 0.192611 |
| 2.13026 | 0.194335 |
| 2.13435 | 0.196059 |
| 2.13645 | 0.197783 |
| 2.13948 | 0.199507 |
| 2.14365 | 0.201232 |
| 2.17106 | 0.202956 |
| 2.17597 | 0.20468 |
| 2.18637 | 0.206404 |
| 2.19104 | 0.208128 |
| 2.19206 | 0.209852 |
| 2.19208 | 0.211576 |
| 2.19228 | 0.2133 |
| 2.19376 | 0.215025 |
| 2.19781 | 0.216749 |
| 2.20296 | 0.222701 |
| 2.20296 | 0.228654 |
| 2.20297 | 0.234606 |
| 2.21208 | 0.23633 |
| 2.21544 | 0.238054 |
| 2.21655 | 0.239778 |
| 2.21899 | 0.245731 |
| 2.22318 | 0.251683 |
| 2.22339 | 0.253407 |
| 2.22738 | 0.255131 |
| 2.22773 | 0.256856 |
| 2.22881 | 0.25858 |
| 2.24017 | 0.260304 |
| 2.24144 | 0.262028 |
| 2.24185 | 0.263752 |
| 2.24309 | 0.269704 |
| 2.24491 | 0.271429 |
| 2.25133 | 0.273153 |
| 2.25425 | 0.274877 |
| 2.25925 | 0.276601 |
| 2.26173 | 0.278325 |
| 2.26333 | 0.280049 |
| 2.267 | 0.281773 |
| 2.26768 | 0.283498 |
| 2.26795 | 0.285222 |
| 2.26929 | 0.286946 |
| 2.27023 | 0.28867 |
| 2.27219 | 0.290394 |
| 2.27819 | 0.292118 |
| 2.28344 | 0.293842 |
| 2.28428 | 0.299795 |
| 2.28656 | 0.301519 |
| 2.29267 | 0.303243 |
| 2.29341 | 0.304967 |
| 2.29488 | 0.306691 |
| 2.298 | 0.308415 |
| 2.30043 | 0.31014 |
| 2.30044 | 0.311864 |
| 2.30134 | 0.313588 |
| 2.3039 | 0.315312 |
| 2.30613 | 0.317036 |
| 2.30681 | 0.31876 |
| 2.32208 | 0.320484 |
| 2.32529 | 0.322209 |
| 2.32529 | 0.323933 |
| 2.3291 | 0.325657 |
| 2.33266 | 0.327381 |
| 2.33549 | 0.329105 |
| 2.3398 | 0.335057 |
| 2.34086 | 0.336782 |
| 2.34181 | 0.338506 |
| 2.34254 | 0.34023 |
| 2.34281 | 0.341954 |
| 2.34374 | 0.347906 |
| 2.34398 | 0.349631 |
| 2.34682 | 0.351355 |
| 2.35016 | 0.353079 |
| 2.35508 | 0.354803 |
| 2.35678 | 0.360755 |
| 2.35788 | 0.362479 |
| 2.35823 | 0.364204 |
| 2.36046 | 0.365928 |
| 2.36239 | 0.367652 |
| 2.36565 | 0.369376 |
| 2.36928 | 0.3711 |
| 2.36999 | 0.372824 |
| 2.37065 | 0.374548 |
| 2.37284 | 0.376273 |
| 2.3748 | 0.377997 |
| 2.37756 | 0.379721 |
| 2.38412 | 0.381445 |
| 2.38493 | 0.383169 |
| 2.39439 | 0.384893 |
| 2.39916 | 0.386617 |
| 2.40431 | 0.39257 |
| 2.40853 | 0.394294 |
| 2.41344 | 0.396018 |
| 2.41348 | 0.397742 |
| 2.41844 | 0.399466 |
| 2.42483 | 0.40119 |
| 2.42523 | 0.402915 |
| 2.42933 | 0.404639 |
| 2.43088 | 0.406363 |
| 2.43223 | 0.408087 |
| 2.4328 | 0.414039 |
| 2.44079 | 0.415764 |
| 2.44218 | 0.417488 |
| 2.44466 | 0.42344 |
| 2.44532 | 0.425164 |
| 2.44635 | 0.426888 |
| 2.44756 | 0.428612 |
| 2.45667 | 0.430337 |
| 2.45862 | 0.432061 |
| 2.46055 | 0.433785 |
| 2.46116 | 0.435509 |
| 2.46332 | 0.437233 |
| 2.46695 | 0.443186 |
| 2.4747 | 0.44491 |
| 2.47567 | 0.446634 |
| 2.47758 | 0.448358 |
| 2.48219 | 0.450082 |
| 2.48234 | 0.451806 |
| 2.48241 | 0.45353 |
| 2.48485 | 0.455255 |
| 2.48626 | 0.461207 |
| 2.50044 | 0.462931 |
| 2.50236 | 0.464655 |
| 2.50725 | 0.466379 |
| 2.51038 | 0.468103 |
| 2.51962 | 0.469828 |
| 2.52071 | 0.47578 |
| 2.52177 | 0.477504 |
| 2.52336 | 0.479228 |
| 2.52745 | 0.480952 |
| 2.52805 | 0.482677 |
| 2.53647 | 0.484401 |
| 2.53818 | 0.490353 |
| 2.53889 | 0.492077 |
| 2.5404 | 0.49803 |
| 2.54461 | 0.499754 |
| 2.54729 | 0.505706 |
| 2.55194 | 0.511658 |
| 2.55317 | 0.513383 |
| 2.55506 | 0.515107 |
| 2.55869 | 0.516831 |
| 2.55939 | 0.522783 |
| 2.56335 | 0.528736 |
| 2.5643 | 0.53046 |
| 2.56624 | 0.532184 |
| 2.56741 | 0.533908 |
| 2.57054 | 0.53986 |
| 2.57123 | 0.541585 |
| 2.57172 | 0.547537 |
| 2.57811 | 0.549261 |
| 2.57832 | 0.550985 |
| 2.58134 | 0.552709 |
| 2.59006 | 0.554433 |
| 2.59101 | 0.556158 |
| 2.59735 | 0.557882 |
| 2.59974 | 0.559606 |
| 2.60041 | 0.565558 |
| 2.60614 | 0.567282 |
| 2.61451 | 0.569007 |
| 2.61565 | 0.570731 |
| 2.61746 | 0.576683 |
| 2.61994 | 0.578407 |
| 2.62066 | 0.580131 |
| 2.62367 | 0.581856 |
| 2.62443 | 0.587808 |
| 2.62498 | 0.589532 |
| 2.63086 | 0.591256 |
| 2.63143 | 0.59298 |
| 2.63486 | 0.594704 |
| 2.6364 | 0.600657 |
| 2.6375 | 0.602381 |
| 2.64482 | 0.604105 |
| 2.65109 | 0.605829 |
| 2.65166 | 0.607553 |
| 2.65259 | 0.609278 |
| 2.65867 | 0.611002 |
| 2.66214 | 0.616954 |
| 2.67811 | 0.618678 |
| 2.68449 | 0.620402 |
| 2.69492 | 0.622126 |
| 2.69503 | 0.623851 |
| 2.69754 | 0.625575 |
| 2.69931 | 0.627299 |
| 2.71209 | 0.629023 |
| 2.71217 | 0.630747 |
| 2.7124 | 0.6367 |
| 2.71395 | 0.638424 |
| 2.71418 | 0.644376 |
| 2.71533 | 0.6461 |
| 2.71891 | 0.647824 |
| 2.72057 | 0.649548 |
| 2.72668 | 0.655501 |
| 2.72668 | 0.657225 |
| 2.73188 | 0.658949 |
| 2.74412 | 0.664901 |
| 2.75143 | 0.666626 |
| 2.75276 | 0.66835 |
| 2.75524 | 0.670074 |
| 2.76211 | 0.671798 |
| 2.76809 | 0.673522 |
| 2.772 | 0.675246 |
| 2.77883 | 0.67697 |
| 2.78093 | 0.682923 |
| 2.78416 | 0.684647 |
| 2.79195 | 0.686371 |
| 2.79394 | 0.688095 |
| 2.79567 | 0.689819 |
| 2.80123 | 0.695772 |
| 2.80468 | 0.701724 |
| 2.80991 | 0.703448 |
| 2.81587 | 0.705172 |
| 2.8214 | 0.706897 |
| 2.82427 | 0.708621 |
| 2.83042 | 0.710345 |
| 2.83594 | 0.716297 |
| 2.83646 | 0.72225 |
| 2.84763 | 0.723974 |
| 2.85731 | 0.729926 |
| 2.8599 | 0.73165 |
| 2.86178 | 0.733374 |
| 2.86706 | 0.739327 |
| 2.87473 | 0.741051 |
| 2.87546 | 0.742775 |
| 2.8819 | 0.748727 |
| 2.89011 | 0.750452 |
| 2.89174 | 0.752176 |
| 2.90023 | 0.758128 |
| 2.91232 | 0.759852 |
| 2.91942 | 0.761576 |
| 2.94519 | 0.7633 |
| 2.96584 | 0.765025 |
| 2.99 | 0.766749 |
| 3.01112 | 0.772701 |
| 3.01422 | 0.774425 |
| 3.01547 | 0.780378 |
| 3.02191 | 0.78633 |
| 3.02659 | 0.792282 |
| 3.04555 | 0.798235 |
| 3.04983 | 0.804187 |
| 3.05026 | 0.81014 |
| 3.05288 | 0.811864 |
| 3.05638 | 0.813588 |
| 3.07232 | 0.815312 |
| 3.07561 | 0.817036 |
| 3.07671 | 0.822989 |
| 3.10018 | 0.828941 |
| 3.11456 | 0.834893 |
| 3.12566 | 0.840846 |
| 3.14601 | 0.84257 |
| 3.1575 | 0.848522 |
| 3.17015 | 0.854475 |
| 3.20958 | 0.860427 |
| 3.23122 | 0.866379 |
| 3.23606 | 0.872332 |
| 3.25956 | 0.874056 |
| 3.26705 | 0.880008 |
| 3.28265 | 0.885961 |
| 3.30849 | 0.887685 |
| 3.35562 | 0.889409 |
| 3.35655 | 0.895361 |
| 3.36746 | 0.901314 |
| 3.37996 | 0.907266 |
| 3.45274 | 0.913218 |
| 3.54148 | 0.919171 |
| 3.54653 | 0.925123 |
| 3.58414 | 0.931076 |
| 3.5889 | 0.9328 |
| 3.5973 | 0.938752 |
| 3.60186 | 0.944704 |
| 3.70092 | 0.950657 |
| 3.70874 | 0.956609 |
| 3.75973 | 0.962562 |
| 3.80078 | 0.968514 |
| 3.82363 | 0.974466 |
| 3.85388 | 0.980419 |
| 3.87584 | 0.982143 |
| 4.04935 | 0.988095 |
| 4.19917 | 0.994048 |

TABLE 2
The look-up table for protein pairs C9 and CK-MB

| Ratio | Score |
|---|---|
| −0.226334 | 0 |
| 0.190081 | 0.00172414 |
| 0.554773 | 0.00344828 |
| 0.897965 | 0.00517241 |
| 1.20613 | 0.00689655 |
| 1.34543 | 0.00862069 |
| 1.54556 | 0.0103448 |
| 1.61083 | 0.012069 |
| 1.67685 | 0.0137931 |
| 1.69013 | 0.0155172 |
| 1.7262 | 0.0172414 |
| 1.76913 | 0.0189655 |
| 1.77609 | 0.0206897 |
| 1.79827 | 0.0224138 |
| 1.87425 | 0.0241379 |
| 1.89609 | 0.0258621 |
| 1.93752 | 0.0275862 |
| 1.94834 | 0.0293103 |
| 2.01464 | 0.0310345 |
| 2.114 | 0.0327586 |
| 2.13581 | 0.0344828 |
| 2.1547 | 0.0362069 |
| 2.18258 | 0.037931 |
| 2.18621 | 0.0396552 |
| 2.21884 | 0.0413793 |
| 2.231 | 0.0431034 |
| 2.23207 | 0.0448276 |
| 2.23848 | 0.0465517 |
| 2.27317 | 0.0482759 |
| 2.27642 | 0.05 |
| 2.31321 | 0.0517241 |
| 2.38288 | 0.0534483 |
| 2.40432 | 0.0551724 |
| 2.40494 | 0.0568966 |
| 2.41653 | 0.0586207 |
| 2.45886 | 0.0603448 |
| 2.47416 | 0.062069 |
| 2.47911 | 0.0637931 |
| 2.49598 | 0.0655172 |
| 2.51081 | 0.0672414 |
| 2.51554 | 0.0689655 |
| 2.51767 | 0.0706897 |
| 2.54119 | 0.0724138 |
| 2.57105 | 0.0741379 |
| 2.58759 | 0.0758621 |
| 2.60991 | 0.0775862 |
| 2.61152 | 0.0835386 |
| 2.63056 | 0.0852627 |
| 2.63114 | 0.0912151 |
| 2.66101 | 0.0929392 |
| 2.70763 | 0.0946634 |
| 2.72322 | 0.0963875 |
| 2.74526 | 0.0981117 |
| 2.74734 | 0.0998358 |
| 2.78355 | 0.10156 |
| 2.81208 | 0.103284 |
| 2.83407 | 0.105008 |
| 2.85908 | 0.106732 |
| 2.85953 | 0.108456 |
| 2.87842 | 0.110181 |
| 2.87885 | 0.111905 |
| 2.90105 | 0.113629 |
| 2.94999 | 0.115353 |
| 2.97974 | 0.117077 |
| 2.98128 | 0.118801 |
| 2.98206 | 0.120525 |
| 3.02152 | 0.12225 |
| 3.02882 | 0.123974 |
| 3.03369 | 0.125698 |
| 3.03993 | 0.127422 |
| 3.04554 | 0.133374 |
| 3.05364 | 0.135099 |
| 3.06558 | 0.136823 |
| 3.08828 | 0.142775 |
| 3.08855 | 0.144499 |
| 3.10403 | 0.146223 |
| 3.12759 | 0.147947 |
| 3.13525 | 0.149672 |
| 3.13874 | 0.151396 |
| 3.15371 | 0.15312 |
| 3.159 | 0.154844 |
| 3.19104 | 0.156568 |
| 3.1935 | 0.158292 |
| 3.20202 | 0.160016 |
| 3.20501 | 0.161741 |
| 3.20518 | 0.163465 |
| 3.21161 | 0.165189 |
| 3.21934 | 0.171141 |
| 3.2194 | 0.172865 |
| 3.23593 | 0.174589 |
| 3.26666 | 0.176314 |
| 3.27394 | 0.178038 |
| 3.28072 | 0.179762 |
| 3.31321 | 0.181486 |
| 3.31506 | 0.18321 |
| 3.32298 | 0.184934 |
| 3.3246 | 0.186658 |
| 3.34283 | 0.188383 |
| 3.346 | 0.190107 |
| 3.34615 | 0.191831 |
| 3.36481 | 0.193555 |
| 3.3679 | 0.195279 |
| 3.37237 | 0.201232 |
| 3.39185 | 0.202956 |
| 3.41818 | 0.20468 |
| 3.41852 | 0.206404 |
| 3.42041 | 0.208128 |
| 3.42362 | 0.209852 |
| 3.42483 | 0.215805 |
| 3.42494 | 0.217529 |
| 3.43788 | 0.219253 |
| 3.44279 | 0.220977 |
| 3.45681 | 0.222701 |
| 3.45839 | 0.224425 |
| 3.47003 | 0.226149 |
| 3.47091 | 0.227874 |
| 3.49556 | 0.229598 |
| 3.5049 | 0.231322 |
| 3.52367 | 0.233046 |
| 3.53754 | 0.23477 |
| 3.54172 | 0.236494 |
| 3.5495 | 0.238218 |
| 3.54975 | 0.239943 |
| 3.5519 | 0.241667 |
| 3.5607 | 0.243391 |
| 3.5634 | 0.245115 |
| 3.56662 | 0.246839 |
| 3.5682 | 0.248563 |
| 3.57013 | 0.250287 |
| 3.57148 | 0.252011 |
| 3.57511 | 0.253736 |
| 3.57576 | 0.25546 |
| 3.58444 | 0.261412 |
| 3.5856 | 0.263136 |
| 3.58629 | 0.26486 |
| 3.59083 | 0.270813 |
| 3.59216 | 0.272537 |
| 3.60223 | 0.274261 |
| 3.60603 | 0.275985 |
| 3.61036 | 0.277709 |
| 3.61155 | 0.279433 |
| 3.61454 | 0.281158 |
| 3.62512 | 0.282882 |
| 3.62703 | 0.284606 |
| 3.65783 | 0.28633 |
| 3.6638 | 0.292282 |
| 3.67064 | 0.294007 |
| 3.67406 | 0.295731 |
| 3.69767 | 0.301683 |
| 3.70958 | 0.303407 |
| 3.71471 | 0.305131 |
| 3.7154 | 0.306856 |
| 3.72936 | 0.30858 |
| 3.72948 | 0.314532 |
| 3.73074 | 0.316256 |
| 3.73099 | 0.31798 |
| 3.73254 | 0.319704 |
| 3.73939 | 0.321429 |
| 3.74602 | 0.323153 |
| 3.74911 | 0.329105 |
| 3.75727 | 0.330829 |
| 3.77279 | 0.332553 |
| 3.78374 | 0.334278 |
| 3.78796 | 0.336002 |
| 3.78874 | 0.341954 |
| 3.79404 | 0.343678 |
| 3.79429 | 0.345402 |
| 3.803 | 0.347126 |
| 3.80348 | 0.348851 |
| 3.81245 | 0.350575 |
| 3.83084 | 0.352299 |
| 3.83375 | 0.354023 |
| 3.83515 | 0.355747 |
| 3.83974 | 0.3617 |
| 3.84208 | 0.363424 |
| 3.84817 | 0.369376 |
| 3.85923 | 0.3711 |
| 3.86075 | 0.372824 |
| 3.87416 | 0.374548 |
| 3.88698 | 0.376273 |
| 3.92448 | 0.382225 |
| 3.92612 | 0.383949 |
| 3.93077 | 0.385673 |
| 3.94494 | 0.387397 |
| 3.94725 | 0.389122 |
| 3.94746 | 0.390846 |
| 3.95203 | 0.39257 |
| 3.95778 | 0.394294 |
| 3.95878 | 0.400246 |
| 3.95944 | 0.406199 |
| 3.97808 | 0.412151 |
| 3.99082 | 0.413875 |
| 3.99246 | 0.419828 |
| 3.99827 | 0.421552 |
| 4.01232 | 0.423276 |
| 4.01258 | 0.425 |
| 4.01738 | 0.426724 |
| 4.02645 | 0.428448 |
| 4.03194 | 0.434401 |
| 4.049 | 0.436125 |
| 4.07491 | 0.437849 |
| 4.08496 | 0.439573 |
| 4.08721 | 0.441297 |
| 4.08771 | 0.443021 |
| 4.08832 | 0.448974 |
| 4.09623 | 0.450698 |
| 4.10251 | 0.452422 |
| 4.10484 | 0.454146 |
| 4.11541 | 0.45587 |
| 4.12058 | 0.457594 |
| 4.1293 | 0.459319 |
| 4.13076 | 0.461043 |
| 4.1312 | 0.466995 |
| 4.13347 | 0.468719 |
| 4.13548 | 0.470443 |
| 4.13552 | 0.472167 |
| 4.14958 | 0.473892 |
| 4.15449 | 0.475616 |
| 4.15704 | 0.47734 |
| 4.15764 | 0.483292 |
| 4.16195 | 0.489245 |
| 4.16367 | 0.490969 |
| 4.16514 | 0.492693 |
| 4.16582 | 0.494417 |
| 4.1671 | 0.496141 |
| 4.17528 | 0.497865 |
| 4.1761 | 0.503818 |
| 4.18212 | 0.505542 |
| 4.18856 | 0.507266 |
| 4.19415 | 0.50899 |
| 4.20375 | 0.510714 |
| 4.20748 | 0.512438 |
| 4.22026 | 0.514163 |
| 4.22941 | 0.515887 |
| 4.24226 | 0.517611 |
| 4.2454 | 0.519335 |
| 4.25104 | 0.521059 |
| 4.25516 | 0.522783 |
| 4.26308 | 0.524507 |
| 4.26695 | 0.526232 |
| 4.27193 | 0.527956 |
| 4.28196 | 0.52968 |
| 4.29025 | 0.531404 |
| 4.29221 | 0.537356 |
| 4.29808 | 0.543309 |
| 4.29963 | 0.545033 |
| 4.304 | 0.546757 |
| 4.30448 | 0.548481 |
| 4.30565 | 0.554433 |
| 4.30905 | 0.556158 |
| 4.31106 | 0.557882 |
| 4.31933 | 0.559606 |
| 4.32344 | 0.56133 |
| 4.32515 | 0.563054 |
| 4.34292 | 0.569007 |
| 4.34485 | 0.570731 |
| 4.34603 | 0.572455 |
| 4.34833 | 0.574179 |
| 4.35987 | 0.575903 |
| 4.37176 | 0.581856 |
| 4.39423 | 0.58358 |
| 4.40203 | 0.585304 |
| 4.42592 | 0.591256 |
| 4.43335 | 0.59298 |
| 4.43683 | 0.594704 |
| 4.4392 | 0.596429 |
| 4.44052 | 0.598153 |
| 4.47806 | 0.599877 |
| 4.48784 | 0.605829 |
| 4.49454 | 0.611782 |
| 4.49587 | 0.613506 |
| 4.50255 | 0.61523 |
| 4.50909 | 0.621182 |
| 4.51629 | 0.622906 |
| 4.51706 | 0.624631 |
| 4.52377 | 0.630583 |
| 4.53237 | 0.636535 |
| 4.55346 | 0.638259 |
| 4.56332 | 0.639984 |
| 4.57978 | 0.645936 |
| 4.59789 | 0.651888 |
| 4.5986 | 0.653612 |
| 4.60803 | 0.659565 |
| 4.61467 | 0.661289 |
| 4.6231 | 0.663013 |
| 4.62674 | 0.664737 |
| 4.63311 | 0.67069 |
| 4.64354 | 0.672414 |
| 4.66008 | 0.674138 |
| 4.6672 | 0.68009 |
| 4.67396 | 0.681814 |
| 4.69528 | 0.683539 |
| 4.70026 | 0.689491 |
| 4.70695 | 0.695443 |
| 4.73734 | 0.697167 |
| 4.73857 | 0.698892 |
| 4.7392 | 0.700616 |
| 4.7573 | 0.70234 |
| 4.75946 | 0.708292 |
| 4.75951 | 0.710016 |
| 4.76245 | 0.711741 |
| 4.76411 | 0.713465 |
| 4.76794 | 0.715189 |
| 4.76798 | 0.721141 |
| 4.78409 | 0.722865 |
| 4.79479 | 0.728818 |
| 4.79762 | 0.730542 |
| 4.79839 | 0.732266 |
| 4.80355 | 0.73399 |
| 4.80624 | 0.739943 |
| 4.8289 | 0.745895 |
| 4.83008 | 0.747619 |
| 4.83868 | 0.749343 |
| 4.84454 | 0.755296 |
| 4.85105 | 0.761248 |
| 4.85985 | 0.762972 |
| 4.86041 | 0.768924 |
| 4.88743 | 0.770649 |
| 4.89332 | 0.772373 |
| 4.89943 | 0.778325 |
| 4.90008 | 0.780049 |
| 4.91305 | 0.786002 |
| 4.92494 | 0.787726 |
| 4.92943 | 0.78945 |
| 4.94202 | 0.791174 |
| 4.94976 | 0.792898 |
| 4.95276 | 0.794622 |
| 4.95422 | 0.796346 |
| 4.95425 | 0.798071 |
| 4.96081 | 0.804023 |
| 4.97422 | 0.805747 |
| 4.97423 | 0.807471 |
| 5.00231 | 0.809195 |
| 5.03683 | 0.81092 |
| 5.04689 | 0.812644 |
| 5.05498 | 0.818596 |
| 5.0612 | 0.82032 |
| 5.08205 | 0.822044 |
| 5.13117 | 0.827997 |
| 5.1335 | 0.829721 |
| 5.13756 | 0.831445 |
| 5.15663 | 0.837397 |
| 5.20514 | 0.839122 |
| 5.21974 | 0.845074 |
| 5.22974 | 0.846798 |
| 5.23944 | 0.85275 |
| 5.27558 | 0.858703 |
| 5.27985 | 0.860427 |
| 5.29093 | 0.866379 |
| 5.32845 | 0.872332 |
| 5.34512 | 0.878284 |
| 5.35676 | 0.884236 |
| 5.38357 | 0.890189 |
| 5.38381 | 0.891913 |
| 5.39237 | 0.893637 |
| 5.40393 | 0.899589 |
| 5.44421 | 0.905542 |
| 5.47254 | 0.911494 |
| 5.51434 | 0.917447 |
| 5.5356 | 0.923399 |
| 5.77057 | 0.929351 |
| 5.8197 | 0.935304 |
| 5.8203 | 0.941256 |
| 6.01465 | 0.94298 |
| 6.01971 | 0.944704 |
| 6.14375 | 0.950657 |
| 6.16432 | 0.956609 |
| 6.23303 | 0.962562 |
| 6.33683 | 0.968514 |
| 6.35384 | 0.974466 |
| 6.4066 | 0.97619 |
| 6.63819 | 0.982143 |
| 6.97235 | 0.988095 |
| 7.11642 | 0.994048 |

As described above, the individual protein pair models vote “progressor” or “control”, and the percentage of pairs within the collection that vote “progressor” provides a score that can be used to assign a sample to the class “progressor” or “control.”

Whether a particular score corresponds to a “progressor” or “control” prediction depends on the “vote threshold”, which can be dialled to tune the sensitivity/specificity. For higher sensitivity at the cost of lower specificity, a vote threshold<50% can be used; for higher specificity at the cost of lower sensitivity, a vote threshold>50% can be used. In this manner, varying the vote threshold to declare a sample as “progressor” may be adjusted to balance sensitivity and specificity as necessary to meet performance objectives and to account for known parameters in a population, such as application within individuals with known HIV-infection.

The invention will be described by way of the following examples which are not to be construed as limiting in any way the scope of the invention.

### EXAMPLES

Methods

Cohorts and Blood Collection

Participants from the South African Adolescent Cohort Study (ACS) were evaluated to identify and validate prospective signatures of risk of tuberculosis disease. The ACS determined the prevalence and incidence of tuberculosis infection and disease among adolescents from the Cape Town region of South Africa (Mahomed, Hawkridge et al. 2011, Mahomed, Ehrlich et al. 2013). A total of 6,363 healthy adolescents, aged 12 to 18 years, were enrolled. Approximately 50% of participants were evaluated at enrolment and every 6 months during 2 years of follow-up; others were evaluated at baseline and at 2 years. At enrolment and at each visit, clinical data were collected, and plasma was collected and stored at −20° C.

In addition, participants from the Grand Challenges 6-74 Study (GC6-74) were studied to independently validate signatures of risk. A total of 4,466 healthy, HIV negative persons aged 10 to 60 years, who had household exposure to an adult with sputum smear positive tuberculosis disease, were enrolled. Sites in South Africa (SUN), the Gambia (MRC), Ethiopia (AHRI) and Uganda participated (ref: http://www.case.edu/affil/tbru/collaborations_gates.html). At baseline and at 6 months (the Gambia only) and at 18 months (all sites), participants were evaluated clinically and blood was collected and plasma isolated and stored at −20° C. Follow-up continued for a total of 2 years.

The study protocols were approved by relevant human research ethics committees. Written informed consent was obtained from participants. For adolescents, consent was obtained from parents or legal guardians of adolescents, and written informed assent from each adolescent.

Definition of Cases and Controls for Identifying and Validating Signatures of Tuberculosis Risk

TB risk signatures were discovered in longitudinally collected plasma from a cohort of M. tuberculosis-infected South Africans adolescents from the ACS where 44 developed microbiologically-confirmed TB disease within two years of follow-up (progressors), and these were matched to 106 non-progressors who remained healthy. Over 3,000 human proteins were quantified with a highly multiplexed proteomic assay (SOMAscan).

Biomarker performance was validated in plasma samples from an independent cohort of GC6-74 adult household contacts (25 progressors and 100 non-progressors) from The Gambia.

Participants with diagnosed or suspected tuberculosis disease were referred to a study-independent public health physician for treatment according to national tuberculosis control programs of the country involved.

Results

The 3-protein pair signature was discovered in a training set of samples for TB progressors and non-progressors. The structure of the 3PR signature is shown in FIG. 1. The performance for predicting TB disease before disease symptoms emerge, stratified by time approaching the onset of TB disease, can be found in FIG. 2.

The 3-protein pair signature was then used for blind validation in a separate cohort of Gambian adult household contacts of TB cases. The blind validation performance can be seen in FIG. 3.

The 3-protein pair signature was found to be capable of predicting TB disease within 2 years before the onset of TB disease symptoms. Prognostic performance of the 3-protein biomarker was AUC=0.894 (95% CI: 0.838-0.95), p<0.0001 on the South African discovery set within 180 days before the diagnosis of TB disease; AUC=0.72 (0.642-0.807), p<0.0001 on the South African discovery set between 180 days and 360 days before the diagnosis of TB disease; and AUC=0.713 (0.631-0.80), p<0.0001 on the South African discovery set between 361 days and 720 days before the diagnosis of TB disease. Prognostic performance of the 3-protein biomarker in the independent validation cohort from The Gambia was AUC=0.65 (0.55-0.75), p=0.0022 within one-year of TB diagnosis in The Gambian validation set (FIG. 3); and AUC=0.64 (0.56-0.73), p=0.0009 within two-year of TB diagnosis in The Gambian validation set.

The signature predicted tuberculosis disease despite multiple confounders, including differences in age range (adolescents versus adults), in infection or exposure status, and in ethnicity and geography between the ACS and GC6-74 cohorts. This result is very encouraging given the distinct genetic backgrounds (Tishkoff, Reed et al. 2009), differing local epidemiology, and differing circulating strains of Mycobacteria (Comas, Coscolla et al. 2013) between South Africa (SUN) and the Gambia (MRC).

A proteomic biomarker of TB risk may allow identification of those who should be investigated for sub-clinical or active TB disease and, if no evidence for disease is found, may benefit from targeted preventive treatment.

### REFERENCES

Comas, I., M. Coscolla, T. Luo, S. Borrell, K. E. Holt, M. Kato-Maeda, J. Parkhill, B. Malla, S. Berg, G. Thwaites, D. Yeboah-Manu, G. Bothamley, J. Mei, L. Wei, S. Bentley, S. R. Harris, S. Niemann, R. Diel, A. Aseffa, Q. Gao, D. Young and S. Gagneux (2013). “Out-of-Africa migration and Neolithic coexpansion of Mycobacterium tuberculosis with modern humans.” Nat Genet 45(10): 1176-1182.
Mahomed, H., R. Ehrlich, T. Hawkridge, M. Hatherill, L. Geiter, F. Kafaar, D. A. Abrahams, H. Mulenga, M. Tameris, H. Geldenhuys, W. A. Hanekom, S. Verver and G. D. Hussey (2013). “TB incidence in an adolescent cohort in South Africa.” PLoS One 8(3): e59652.
Mahomed, H., T. Hawkridge, S. Verver, D. Abrahams, L. Geiter, M. Hatherill, R. Ehrlich, W. A. Hanekom and G. D. Hussey (2011). “The tuberculin skin test versus QuantiFERON TB Gold(R) in predicting tuberculosis disease in an adolescent cohort study in South Africa.” PLoS One 6(3): e17984.
Owzar, K., W. T. Barry and S. H. Jung (2011). “Statistical considerations for analysis of microarray experiments.” Clin Trans! Sci 4(6): 466-477.
Platt, J. C. (1998). “Sequential Minimal Optimization: A Fast Algorithm for Training Support Vector Machines.” Microsoft Research Technical Report MSR-TR-98-14.
Sambrook, J., D. W. Russell and J. Sambrook (2006). The condensed protocols from Molecular cloning: a laboratory manual. Cold Spring Harbor, N.Y., Cold Spring Harbor Laboratory Press.
Shi, P., S. Ray, Q. Zhu and M. A. Kon (2011). “Top scoring pairs for feature selection in machine learning and applications to cancer outcome prediction.” BMC Bioinformatics 12: 375.
Tishkoff, S. A., F. A. Reed, F. R. Friedlaender, C. Ehret, A. Ranciaro, A. Froment, J. B. Hirbo, A. A. Awomoyi, J. M. Bodo, O. Doumbo, M. Ibrahim, A. T. Juma, M. J. Kotze, G. Lema, J. H. Moore, H. Mortensen, T. B. Nyambo, S. A. Omar, K. Powell, G. S. Pretorius, M. W. Smith, M. A. Thera, C. Wambebe, J. L. Weber and S. M. Williams (2009). “The genetic structure and history of Africans and African Americans.” Science 324(5930): 1035-1044.
Wang, Z., M. Gerstein and M. Snyder (2009). “RNA-Seq: a revolutionary tool for transcriptomics.” Nat Rev Genet 10(1): 57-63.
WHO, W. H. O. (2014) “Global Tuberculosis Report 2014.”.

1-18. (canceled)

19. A kit comprising at least three protein biomarker capture reagents, wherein each protein biomarker capture reagent specifically binds to a protein target from a subject selected from the group consisting of C9, C1qTNF3 and CK-MB, and wherein each protein biomarker capture reagent specifically binds to a different target protein.

20. The kit of claim 19, wherein the capture reagents comprise three aptamers or antibodies, wherein each aptamer or antibody specifically binds to a different target protein.

21. The kit of claim 19, wherein the capture reagents are labeled with an indicator molecule including a fluorescent, chemiluminescent, radioactive, or chromogenic molecule

22. The kit of claim 19, further comprising one or more of: a solid support, instructions for use of the kit, a computer system or software to analyze data, and additional reagents for quantifying the levels of the protein biomarkers including reagents for processing a biological sample including solubilization buffers, detergents, washes, or buffers, and buffers, blocking agents, mass spectrometry matrix materials, antibody capture agents, detectable labels, signal generating material, positive control samples and negative control samples.

23. The kit of claim 22, wherein the instructions for use of the kit include instructions for monitoring a latent TB infection in a subject for the likelihood of the latent TB infection transitioning to active TB disease comprising:
a) quantifying and computationally analysing relative abundances of a 3 protein pair-ratio (3PR) signature consisting of a first and a second pair of proteins selected from Complement Component 9 (C9) and Complement C lq Tumor Necrosis Factor-Related Protein 3 (C1qTNF3), and C9 and Creatine Kinase M- and B-type (CKMB); and
b) computing a prognostic score of the risk of the subject developing active TB disease, thus classifying the subject as “progressor” or “non-progressor”, wherein a prognostic score of “progressor” indicates that the subject with asymptomatic TB infection or suspected TB infection is likely to progress to active tuberculosis disease.

24. The kit of claim 23, wherein computationally analysing comprises:
(i) the computation of a log ratio:
r=log_2(concentration protein 1/concentration protein 2) for the first and second pair of proteins from the sample; and
(ii) use of a score table that has been calculated by analysis of a prospective TB risk cohort to convert the computed log ratio for each pair of proteins into a score;
(iii) followed by calculation of the mean final score from both pairs of proteins from the sample,
wherein the mean final score is predictive of the likelihood of the subject with asymptomatic TB infection or suspected TB infection progressing to active tuberculosis disease.

25. The kit of claim 24, wherein computationally analysing comprises:
A. quantifying the protein concentration of the three proteins C9, C1qTNF3 and CK-MB;
B. computing the difference in concentration between the protein pairs C9 and C 1qTNF3 (pair 1) and C9 and CK-MB (pair 2) to generate a log-transformed ratio of expression for each pair;
C. comparing the log-transformed ratio of expression for pair 1 and pair 2 to the closest minimal ratios listed in Table 1 and Table 2 respectively by finding the minimal ratio in the first column of the respective table that is greater than or equal to the computed log-transformed ratio;
D. assigning a corresponding numerical score in the second column of the respective table to the computed log-transformed ratio for pair 1 and pair 2, wherein if the computed log-transformed ratio is greater than all of the ratios in column 1 of the respective table, assigning a numerical score of 1 to the computed log-transformed ratio; and
E. determining the final score for pair 1 and pair 2 by computing the average value of the numerical scores generated from both pair 1 and pair 2,
wherein the final score is predictive of the likelihood of the subject with asymptomatic TB infection or suspected TB infection progressing to active tuberculosis disease.

26. The kit of claim 25, wherein the default threshold for progressor versus non-progressors is 0.5 (i.e. 50%).

27. The kit of claim 25, wherein the default threshold for progressor versus non-progressors is greater than 0.5 (i.e. 50%).

28. The kit of claim 25, wherein the default threshold for progressor versus non-progressors is less than 0.5 (i.e. 50%).

29. The kit of claim 22, wherein the computer system or software to analyze data comprises computer readable instructions for performing each of the steps of the computational analysis.

30. The kit of claim 19, wherein the target proteins are indicative of one or more of: the presence of latent TB infection, the presence of active TB disease, the strain of TB, the antibiotic resistance or sensitivity of TB, and the presence of non-TB diseases.

31. The kit of claim 19, wherein the subject is identified as being likely to transition to active TB disease within 30 days, 45 days, 60 days, 90 days, 120 days, 180 days, 270 days, 360 days, 450 days, or 540 days if a prognostic score of “progressor” is computed.

32. The kit of claim 22, wherein the instructions for use comprise directing the use of any one or more techniques including lateral flow technology, enzyme-linked immunosorbent assay (ELISA), surface plasmon resonance, surface acoustic waves, mass spectrometry, infrared spectroscopy, Raman spectroscopy, atomic force microscopy, scanning tunneling microscopy, electrochemical detection methods, nuclear magnetic resonance or quantum dots.

33. An admixture of three aptamers or antibodies, wherein each aptamer or antibody specifically binds to the protein biomarkers C9, C1qTNF3 or CK-MB respectively.

34. The admixture according to claim 33, wherein each aptamer or antibody is labeled with an indicator molecule including a fluorescent, chemiluminescent, radioactive, or chromogenic molecule.

35. A composition comprising target proteins in a sample from a subject and three protein biomarker capture reagents, wherein each protein biomarker capture reagent specifically binds to a target protein selected from the group consisting of a first and a second pair of protein biomarkers selected from Complement Component 9 (C9) and Complement C1q Tumor Necrosis Factor-Related Protein 3 (C1qTNF3); and C9 and Creatine Kinase M- and B-type (CKMB), and wherein each protein biomarker capture reagent specifically binds a different target protein.

36. The composition of claim 35, wherein at least one biomarker capture reagent includes an antibody or aptamer.

37. The composition of claim 35, wherein the sample is one or more biological material sample(s) derived from a human, including a blood sample, a blood plasma sample, a blood serum sample derived from clotted whole blood, a blood protein sample, a sputum sample, a sputum protein sample, a urine sample, a saliva sample, a cerebrospinal fluid sample, a pleural effusion sample, a pericardial effusion sample, a tissue aspirate, or a biopsy sample.

38-47. (canceled)

48. A surface comprising three aptamers or antibodies, wherein each aptamer or antibody specifically binds to the protein biomarkers C9, C1qTNF3 and CK-MB respectively.

49. The surface according to claim 48, wherein each aptamer or antibody is labeled with an indicator molecule selected from a fluorescent, chemiluminescent, radioactive, and chromogenic molecule.
